(19) United States
(12) Patent Application Publication
(10) Pub. No.: US 2009/0203676 A1
(43) Pub. Date: Aug. 13, 2009

(54) G-protein Coupled Receptor Agonists

(76) Inventors: Oscar Barba, Oxford (GB); Stuart Edward Bradley, Oxford (GB); Matthew Colin Thor Fyfe, Oxford (GB); Lisa Sarah Bertram, Oxford (GB); William Gattrell, Oxford (GB); Martin James Procter, Oxford (GB); Chrystelle Marie Rasamison, Oxford (GB); Simon Andrew Swain, Oxford (GB)

Correspondence Address: OSI PHARMACEUTICALS, INC., 41 PINELAWN ROAD, MELVILLE, NY, 11747, US

(21) Appl. No.: 11/922,826

(22) PCT Filed: Jun. 30, 2006
(86) PCT No.: PCT/GB2006/050182

§ 371 Date: Jan. 30, 2009

(30) Foreign Application Priority Data
Jun. 30, 2005 (GB) 0513276.6
Jun. 29, 2006 (GB) 0612897.9

(51) Int. Cl. A61K 31/496; C07D 401/06; C07D 401/14; C07D 413/14; C07D 497/08; A61K 31/551; A61K 31/454; A61P 3/04; A61P 3/10; A61P 3/00

(52) U.S. Cl. 514/218; 544/360; 540/575; 546/199; 514/253.13; 514/253.09; 514/253.11; 514/253.1; 514/322

(57) ABSTRACT

Compounds of formula (I): or pharmaceutically acceptable salts thereof, are GPCR agonists and are useful as for the treatment of obesity and diabetes.

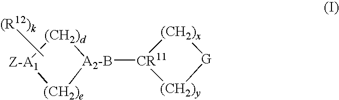

The present invention is directed to G-protein coupled receptor (GPCR) agonists. In particular, the present invention is directed to GPCR agonists that are useful for the treatment of obesity, e.g. as regulators of satiety, and for the treatment of diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent application WO2005/061489 (published after the priority date of the present application) discloses heterocyclic derivatives as GPR119 receptor agonists.

The present invention relates to agonists of GPR119 which are useful for the treatment of obesity e.g. as peripheral regulators of satiety, and for the treatment of diabetes.

### SUMMARY OF THE INVENTION

Compounds of formula (I):

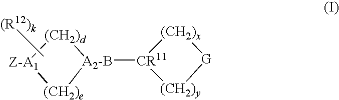

or pharmaceutically acceptable salts thereof, are agonists of GPR119 and are useful for the prophylactic or therapeutic treatment of obesity and diabetes.

### DETAILED DESCRIPTION OF THE INVENTION

The present invention is directed to a compound of formula (I):

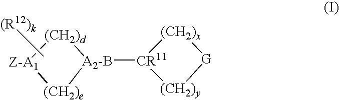

or a pharmaceutically acceptable salt thereof, wherein:

Z represents an aryl, heteroaryl, —C₁₋₄alkylaryl or —C₁₋₄alkylheteroaryl group, any of which may optionally be substituted by one or more groups selected from halogen, C₁₋₄ alkyl, C₁₋₄ fluoroalkyl, C₁₋₄ hydroxyalkyl, C₂₋₄ alkenyl, C₂₋₄ alkynyl, C₁₋₄alkoxy, OR⁹, NR³R⁴, S(O)ₙR⁹, S(O)₂NR⁹R⁹⁹, C(O)NR⁹R⁹⁹, NR¹⁰C(O)R⁹, NR¹⁰C(O)NR⁹R⁹⁹, NR¹⁰SO₂R⁹, C(O)R⁹, C(O)OR⁹, —P(O)(CH₃)₂, NO₂, cyano or —(CH₂)ⱼ—C₃₋₇cycloalkyl, —(CH₂)ⱼ-aryl, —(CH₂)ⱼ-heterocyclyl, —(CH₂)ⱼ-heteroaryl, any of which cycloalkyl, aryl, heterocyclyl or heteroaryl groups may be substituted by C₁₋₄alkyl;

one of A₁ and A₂ is N or N⁺—O⁻, and the other is CH, C(OH) or N;

d is 0, 1, 2, or 3;

e is 1 or 2;

with the proviso that d+e is 2, 3, 4 or 5, and that if A₁ and A₂ are both N, d is 2 or 3 and e is 2;

j is 0, 1 or 2;

k is 0, 1 or 2;

n is 0, 1, or 2;

B represents a branched or unbranched C₁₋₄alkylene chain or C₁₋₄alkenylene chain, either of which may optionally be substituted by one or more groups selected from halogen, hydroxy or oxo, and wherein one CH₂ group may be replaced by O or NR⁸, provided that the group >A₂-B— does not contain any direct N—O, N—C—O, N—N, N—C—N or N—C-halogen bonds;

G represents CHR² or NR¹;

R¹ is C(O)OR⁵, C(O)R⁵, S(O)₂R⁵, C(O)NR⁵R⁸, C₁₋₄alkylene-C(O)OR⁵, C(O)C(O)OR⁵, or P(O)(O-Ph)₂; or heterocyclyl or heteroaryl, either of which may optionally be substituted by one or two groups selected from C₁₋₄alkyl, C₁₋₄alkoxy or halogen;

R² is C₃₋₆alkyl;

R³ and R⁴ are independently hydrogen, methoxy, C₁₋₄ alkyl, which may optionally be substituted by halo (e.g. fluoro), hydroxy, C₁₋₄ alkyloxy-, aryloxy-, arylC₁₋₄ alkyloxy-, C₁₋₄ alkylS(O)ₙ—, C₃₋₇heterocyclyl, —C(O)OR¹⁴ or N(R¹⁰)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR¹³, CN, SO₂CH₃, N(R¹⁰)₂ and NO₂; or taken together R³ and R⁴ may form a 5- or 6-membered heterocyclic ring optionally substituted by hydroxy, C₁₋₄ alkyl or C₁₋₄ hydroxyalkyl and optionally containing a further heteroatom selected from O and NR¹⁰;

R⁵ and R⁵⁵ are independently C₁₋₈ alkyl, C₂₋₈ alkenyl or C₂₋₈ alkynyl, any of which may be optionally substituted by one or more halo atoms, NR⁶R⁶⁶, OR⁶, C(O)OR⁶, OC(O)R⁶ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyeneheterocyclyl or C₁₋₄ alkyleneheteroaryl, any of which may be substituted with one or more substituents selected from halo, C₁₋₄ alkyl, C₁₋₄ fluoroalkyl, OR⁷, CN, NR⁷R⁷⁷, SO₂Me, NO₂ or C(O)OR⁷;

R⁶, R⁶⁶, R⁷, and R⁷⁷ each independently are hydrogen or C₁₋₄alkyl; or, taken together, R⁶ and R⁶⁶ or R⁷ and R⁷⁷ may independently form a 5- or 6-membered heterocyclic ring;

R⁸ hydrogen or C₁₋₄alkyl;

R⁹ and R⁹⁹ are independently hydrogen, methoxy, C₁₋₄ alkyl, which may optionally be substituted by halo (e.g. fluoro), hydroxy, C₁₋₄ alkoxy-, C₁₋₄ alkoxyC₁₋₄ alkoxy-, -aryloxy-, arylC₁₋₄ alkyloxy-, C₁₋₄ alkylS(O)ₙ—, C₃₋₇ heterocyclyl, —C(O)OR¹⁴ or N(R¹⁰)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR¹³, CN, SO₂CH₃, N(R¹⁰)₂ and NO₂; or taken together R⁹ and R⁹⁹ may form a 5- or 6-membered heterocyclic ring optionally substituted by hydroxy, C₁₋₄ alkyl or C₁₋₄ hydroxyalkyl and optionally containing a further heteroatom selected from O and NR¹⁰;

R¹⁰ is hydrogen, C₁₋₄alkyl; or a group N(R¹⁰)₂ may form a 4- to 7-membered heterocyclic ring optionally containing a further heteroatom selected from O and NR¹⁰;

R¹¹ is hydrogen or hydroxy, or when B represents C₁₋₄alkenylene and there is a point of unsaturation adjacent to CR¹¹ then R¹¹ is absent;

R¹² is each independently hydroxy, oxo, methyl; or two R¹² groups may form a bridging methylene;

R¹³ is hydrogen, C₁₋₂ alkyl or C₁₋₂ fluoroalkyl;

R¹⁴ is hydrogen or C₁₋₄ alkyl;

x is 0, 1, 2 or 3; and

y is 1, 2, 3, 4 or 5;

with the proviso that x+y is 2, 3, 4 or 5.

The molecular weight of the compounds of formula (I) is preferably less than 800, more preferably less than 600, even more preferably less than 500.

One group of compounds of interest are those of formula (Ia):

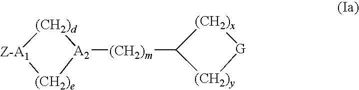

or a pharmaceutically acceptable salt thereof, wherein:

Z represents an aryl or heteroaryl group, either of which may optionally be substituted by one or more groups selected from halogen, C₁₋₄alkoxy, NR³R⁴, S(O)ₘR⁹, S(O)₂NR⁹R⁹⁹ C(O)NR⁹R⁹⁹, C(O)R⁹, C(O)OR⁹, aryl, heterocyclyl, heteroaryl or cyano; or C₁₋₄alkyl, C₂₋₄alkenyl, or C₂₋₄alkynyl any of which three may optionally be substituted by one or more halogen, hydroxy, NR³R⁴, oxo or C₁₋₄alkoxy;

one of A₁ and A₂ is N, and the other is CH or N;

d is 0, 1, 2, or 3;

e is 1 or 2;

with the proviso that d+e is 2, 3, 4 or 5, and that if A₁ and A₂ are both N, d is 2 or 3 and e is 2;

m is 1, 2 or 3;

G represents CHR² or NR¹;

R¹ is C(O)OR⁵, C(O)R⁵, S(O)₂R⁵, C(O)NR⁵R⁸, C₁₋₄alkylene-C(O)OR⁵, C(O)C(O)OR⁵, S(O)₂R⁵, C(O)R⁵ or P(O)(O-Ph)₂; or heterocyclyl or heteroaryl, either of which may optionally be substituted by one or two groups selected from C₁₋₄alkyl, C₁₋₄alkoxy or halogen;

R² is C₃₋₆ alkyl;

R³ and R⁴ are independently hydrogen, C₁₋₄alkyl, C₃₋₇cycloalkyl, or aryl, which may optionally be substituted with 1 or 2 substituents selected from halo, C₁₋₄alkyl, CF₃, C₁₋₄alkoxy, cyano, and S(O)₂Me; or, taken together, R⁴ and R⁴⁴ may form a 5- or 6-membered heterocyclic ring;

R⁵ and R⁵⁵ are independently C₁₋₈ alkyl, C₂₋₈ alkenyl or C₂₋₈ alkynyl, any of which may be optionally substituted by one or more halo atoms, NR⁶R⁶⁶, OR⁶, C(O)OR⁶, OC(O)R⁶ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyeneheterocyclyl or C₁₋₄ alkyleneheteroaryl, any of which may be substituted with one or more substituents selected from halo, C₁₋₄ alkyl, C₁₋₄ fluoroalkyl, OR⁷, CN, NR⁷R⁷⁷, SO₂Me, NO₂ or C(O)OR⁷;

R⁶, R⁶⁶, R⁷, and R⁷⁷ each independently are hydrogen or C₁₋₄alkyl; or, taken together, R⁶ and R⁶⁶ or R⁷ and R⁷⁷ may independently form a 5- or 6-membered heterocyclic ring;

R⁸ hydrogen or C₁₋₄alkyl;

R⁹ and R⁹⁹ are independently hydrogen, C₁₋₄ alkyl, which may optionally be substituted by halo (e.g. fluoro), hydroxy, C₁₋₄ alkyloxy-, C₁₋₄ alkylthio-, C₃₋₇ heterocyclyl or N(R¹⁰)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁹, CN, SO₂CH₃, N(R¹⁰)₂ and NO₂;

x is 0, 1, 2 or 3; and

y is 1, 2, 3, 4 or 5;

with the proviso that x+y is 2, 3, 4 or 5.

Exemplary aryl groups which Z may represent include phenyl and naphthalenyl (either of which may be optionally substituted as described above), in particular phenyl. Exemplary heteroaryl groups which Z may represent include 5 membered monocyclic rings, 6 membered monocyclic rings, 8 membered bicyclic rings, 9 membered bicyclic rings and 10 membered bicyclic rings (any of which may be optionally substituted as described above), in particular 6 membered monocyclic rings (such as those containing one or two nitrogen atoms). When Z is a heteroaryl or —C₁₋₄alkylheteroaryl group it will typically contain up to four heteroatoms selected from O, N and S. When Z represents —C₁₋₄alkylaryl or —C₁₋₄alkylheteroaryl, it is suitably —C₁₋₂alkylaryl or —C₁₋₂alkylheteroaryl.

Z is preferably phenyl or 6-membered heteroaryl preferably containing one nitrogen atom.

When Z is a substituted phenyl or a 6-membered heteroaryl group containing one nitrogen atom it is preferably substituted by up to 3 substituents preferably in the meta and para positions.

Preferred groups by which Z may be substituted include S(O)ₙR⁹ e.g. SOMe or SO₂Me, C(O)NR⁹R⁹⁹, NR¹⁰C(O)NR⁹R⁹⁹, 5- or 6-membered heteroaryl, halogen e.g. fluoro or chloro, C₁₋₄ alkyl e.g. methyl and cyano.

G is preferably NR¹.

R¹ is preferably C(O)OR⁵, C(O)NR⁵R⁸, C₁₋₄alkylene-C(O)OR⁵, C(O)C(O)OR⁵, heterocyclyl, heteroaryl, S(O)₂R⁵, C(O)R⁵ or P(O)(O-Ph)₂; especially C(O)OR⁵, C(O)NR⁵R⁸, C₁₋₄alkyl-C(O)OR⁵, heteroaryl, S(O)₂R⁵ or C(O)R⁵; in particular C(O)OR⁵, C(O)NR⁵R⁸, heteroaryl, S(O)₂R⁵ or C(O)R⁵. More preferably, R¹ is C(O)OR⁵, C(O)NR⁵R⁸ or heteroaryl. R¹ is most preferably COOR⁵. When R¹ is heteroaryl the heteroaryl ring is preferably a 5- or 6-membered heteroaryl ring, for example pyrimidinyl, especially pyrimidin-2-yl.

Preferably R⁵ represents C₁₋₈ alkyl, C₂₋₈ alkenyl or C₂₋₈ alkynyl optionally substituted by one or more halo atoms or cyano, and which may contain a CH₂ group that is replaced by O or S; or a C₃₋₇cycloalkyl, aryl or C₁₋₄alkylC₃₋₇ cycloalkyl, any of which may be substituted with one or more substituents selected from halo, C₁₋₄ alkyl, C₁₋₄ fluoroalkyl, OR⁷, CN, NR⁷R⁷⁷, NO₂ and C(O)OC₁₋₄alkyl. More preferably R⁵ represents C₁₋₈alkyl, C₂₋₈ alkenyl or C₂₋₈ alkynyl optionally substituted by one or more halo atoms or cyano, and which may contain a CH₂ group that is replaced by O or S; or a C₃₋₇cycloalkyl or aryl, either of which may be substituted with one or more substituents selected from halo, C₁₋₄ alkyl, C₁₋₄ fluoroalkyl, OR⁷, CN, NR⁷R⁷⁷, NO₂ and C(O)OC₁₋₄alkyl. Most preferred R⁵ groups are C₃₋₅alkyl optionally substituted by one or more halo atoms or cyano, and may contain a CH₂ group that is replaced by O or S; or C₃₋₅cycloalkyl optionally substituted by C₁₋₄ alkyl. In one embodiment of the invention the group represented by R⁵ is unsubstituted.

In one embodiment of the invention x+y is 2, 3, or 4. In a preferred embodiment of the invention x and y each represent 1. In a more preferred embodiment of the invention x and y each represent 2.

Suitably B represents a branched or unbranched C₁₋₄alkylene which may optionally be substituted by one or more groups selected from halogen, hydroxy or oxo. Alternatively B represents a branched or unbranched C₁₋₄alkenylene which may optionally be substituted by one or more groups selected from halogen, hydroxy or oxo. When the group B is substituted, suitably it is substituted by 1, 2 or 3 substituent groups (e.g. 1 or 2).

In one embodiment of the invention A₁ and A₂ represent N. In a second embodiment of the invention A₁ represents N and A₂ represents CH. In a third embodiment of the invention A₁ represents CH and A₂ represents N.

A subgroup of compounds of formula (I) of are those of formula (Ib):

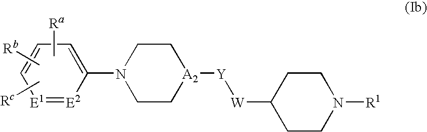

wherein E¹ and E² are CH, or one of E¹ and E² is n and the other is CH;

A₂ is N or CH;

when A₂ is N, Y is CH₂;

when A₂ is CH, Y is O or NR⁸;

W is a branched or unbranched C₁₋₃alkylene chain or C₁₋₃alkenylene chain, either of which may optionally be substituted by one or more groups selected from halogen, hydroxy or oxo;

one of R^{a}, R^{b} and R^{c} is selected from S(O)ₙR⁹, S(O)₂NR⁹R⁹⁹, C(O)NR⁹R⁹⁹, NR¹⁰C(O)NR⁹R⁹⁹ and 5- or 6-membered heteroaryl, and the other two of R^{a}, R^{b} and R^{c} are selected from hydrogen, halogen, C₁₋₄ alkyl and cyano; and

R¹ is C(O)OR⁵, C(O)NR⁵R⁸ or 5- or 6-membered heteroaryl.

For the avoidance of doubt in the CH group represented by E¹ or E² the H may be replaced by one of the substituents listed above for R^{a}, R^{b} and R^{c}.

In the compounds of formula (Ib) one of E¹ or E² is preferably N.

While the preferred groups for each variable have generally been listed above separately for each variable, preferred compounds of this invention include those in which several or each variable in formulae (I), (Ia) and (Ib) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

The following provisos may optionally be used (individually or in any combination) to exclude certain compounds from the scope of the invention:

i) when G represents N—C(O)O-tert-butyl; B represents an ethylene group; A₁ and A₂ each represent N; d, e, x, and y each represent 2; R¹¹ represents H; k represents 0; suitably Z does not represent:

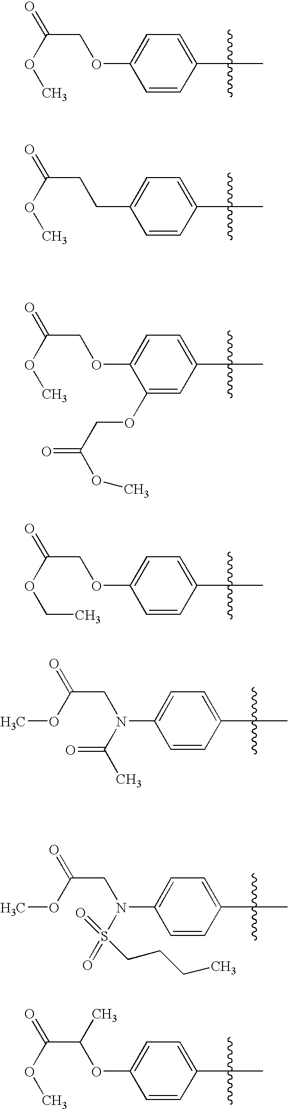

ii) when Z represents phenyl; B represents a methylene group; A₁ represent CH; A₂ represents N; d, e, x, and y each represent 2; R¹¹ represents H; k represents 0; suitably G does not represent:

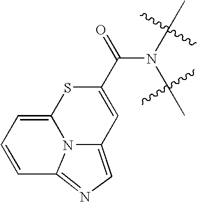

iii) when G represents N-(naphthylen-1-ylsulphonyl-); B represents an ethylene group; A₁ and A₂ each represent N, or A₁ represents CH and A₂ represents N; d and e each represent 2; x represents 0; y represents 4; R¹¹ represents H; k represents 0; suitably Z does not represent phenyl, pyridine-2-yl-, 2-methylphenyl-, 4-trifluoromethylphenyl- or 3-trifluoromethylphenyl.

iv) when G represents N-4-trifluoromethylphenylsulphonyl-); B represents a methylene group; A₁ represents CH and A₂ represents N; d, e, x and y each represent 2; R¹¹ represents H; k represents 0; suitably Z does not represent pyridine-5-yl-.

v) when Z represents 2-methoxyphenyl-; B represents a methylene group; A₁ and A₂ represent N; d and e each represent 2; x represents 1 and y represents 3, or x represents 2 and y represents 2; R¹¹ represents H; k represents 0; suitably G does not represent N—C(O)-phenyl or N—C(O)-cyclohexyl.

vi) when B represents a methylene group; A₁ and A₂ represent N; d and e each represent 2; x represents 1 and y represents 3, or x represents 2 and y represents 2; R¹¹ represents H; k represents 0; Z represents 3-(dimethylamino)phenyl-, 3-(acetamido)phenyl-, 2-methoxyphenyl-, pyrid-2-yl, suitably G does not represent N-(4-methylphenylsulphonyl-), N-(4-fluorophenylsulphonyl-) or N-(cyclohexylmethanesulphonyl-).

As used herein, unless stated otherwise, “alkyl” as well as other groups having the prefix “alk” such as, for example, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The term “cycloalkyl” means carbocycles containing no heteroatoms, and includes monocyclic and bicyclic saturated and partially saturated carbocycles. Examples of cycloalkyl include cyclopropyl, cyclobutyl, cyclopentyl, cyclohexyl and cycloheptyl. Examples of partially saturated cycloalkyl groups include cyclohexene and indane. Cycloalkyl groups will typically contain 3 to 10 ring carbon atoms in total (e.g. 3 to 6, or 8 to 10).

The term “halo” includes fluorine, chlorine, bromine, and iodine atoms (in particular fluorine or chlorine).

The term “aryl” includes phenyl and naphthyl, in particular phenyl.

Unless otherwise indicated the term “heterocyclyl” and “heterocyclic ring” includes 4- to 10-membered monocyclic and bicyclic saturated rings, e.g. 4- to 7-membered monocyclic saturated rings, containing up to three heteroatoms selected from N, O and S. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered, e.g. monocyclic 5- or 6-membered, heteroaryl rings containing up to 4 heteroatoms selected from N, O and S. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine. Preferred heteroaryl groups are monocyclic 5- or 6-membered, heteroaryl rings containing up to 4 heteroatoms selected from N, O and S.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise. For example the invention includes all keto and enol forms which may be encompassed by the definition of B.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein the groups Z, A¹, A², R¹¹, R¹², d, e, m, x, y, and G are as defined above.

Compounds of formula (I) in which A₂ is N may be prepared as described in Scheme 1 by reductively alkylating the amine 2 with the aldehyde 3 where Bₓ, represents B minus CH₂, employing a suitable reductant, e.g., sodium triacetoxyborohydride (Abdel-Magid, A. F., et al., J. Org. Chem. 1996, 61, 3849-3862), in an appropriate solvent, e.g., dichloromethane, at around 20° C. The aldehydes 3, as well as the amines 2, are either commercially available or are made easily using known techniques.

Compounds of formula (I) where B contains a NR⁸ group may also be prepared by reductive alkylations of this type using appropriate intermediates, for examples compounds corresponding to those of formula 2 where instead of NH A₂ represents >CH—NH₂.

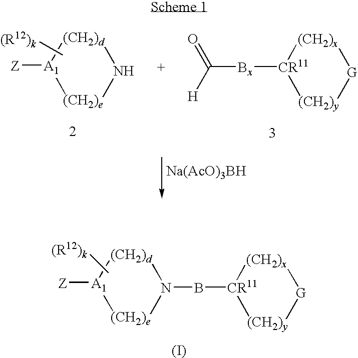

Compounds of formula (I) may be prepared from amine 4 and compound 5 where L is a leaving group such as mesylate/tosylate/halide with triethylamine, DIPEA or potassium carbonate. Where R¹² is oxo and adjacent to A₂, sodium hydride is used as the base.

Compounds of formula (I) where B contains an O group may also be prepared by similar methods using appropriate intermediates, for examples compounds corresponding to those of formula 2 where instead of NH A₂ represents >CH—OH.

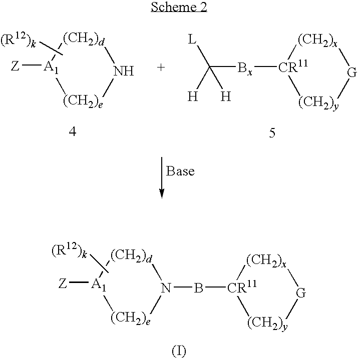

Compounds of the formula (I) may also be prepared from lithium halogen exchange with bromide of Z followed by nucleophilic attack on the cyclic ketone 6 as shown in Scheme 3. Alternative organometallics may be used, e.g. ZMgX.

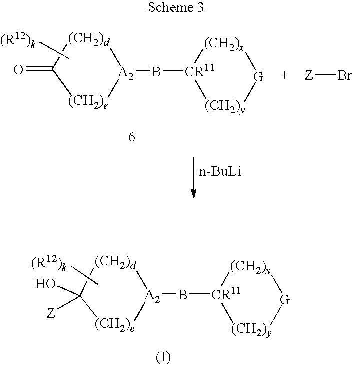

Compounds of the formula (I) may also be prepared by coupling amines 7 and carboxylic acids 8 to give amide examples as shown in Scheme 4.

The chemistry in Scheme 4 can also be used to prepare examples where A₂ is CH or N and B is a linker containing an amide moiety.

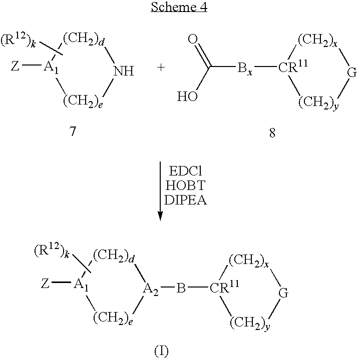

Compounds of the formula (I) where B is alkenylene may also be prepared using the Wittig reaction from ketone 9 and phosphonium salt 10 as shown in Scheme 5. Further modification can be carried out by hydrogenation using a suitable catalyst, e.g. Pd on carbon, to give the saturated analogue of formula (I).

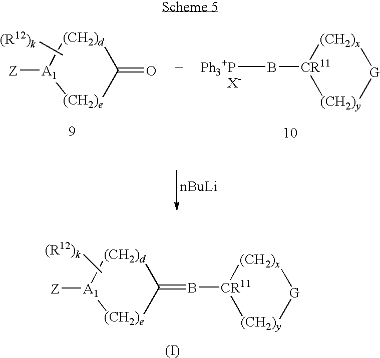

Compounds of formula (I) in which R¹ is C(O)OR⁵, C(O)R⁵, S(O)₂R⁵, C(O)NR⁵R⁵⁵, or heteroaryl may be prepared by the route shown in Scheme 2. Compounds of formula 4, in which PG represents a suitable protecting group, for example tert-butoxycarbonyl (Boc), may be synthesised as outlined above. The protecting group is firstly removed under suitable conditions to afford compounds of formula 12. In the case of the Boc group this can be achieved by treatment of compounds of formula 11 with a suitable acid, such as trifluoroacetic acid (Fyfe, M. C. T. et al. International Patent Publication WO 04/72031), in an appropriate solvent, such as CH₂Cl₂. Treatment of compounds of formula 12 with chloroformates Cl—R¹, which are generally commercially available or can be readily synthesised, in a suitable solvent, such as CH₂Cl₂, in the presence of a suitable base, such as triethylamine (Picard, F., et al. J. Med. Chem. 2002, 45, 3406-3417), affords compounds of formula (I) where R¹ is C(O)OR⁵. Similarly, compounds of formula 17 may be reacted with sulfonyl chlorides, carboxylic acid chlorides, and carbamyl chlorides Cl—R¹, which are generally commercially available or can readily be synthesised, in a suitable solvent, such as CH₂Cl₂, in the presence of a suitable base, such as triethylamine, to afford compounds of formula (I) where R¹ is S(O)₂R⁵, C(O)R⁵, and C(O)NR⁵R⁸, respectively. Furthermore, compounds of formula (I) in which R¹ is heteroaryl may be prepared by reacting the amine 12 with the appropriate heteroaryl chloride or bromide under Pd(0) catalysis in the presence of a suitable ligand and base (Urgaonkar, S.; Hu, J.-H.; Verkade, J. G. J. Org. Chem. 2003, 68, 8416-8423). Alternatively, compounds of the formula (I) where R¹ is heteroaryl may be prepared by condensation of amine 17 with a heteroaryl chloride in the presence of base (Barillari, C. et al. Eur. J. Org. Chem. 2001, 4737-4741; Birch, A. M. et al. J. Med. Chem. 1999, 42, 3342-3355). Compounds of formula (I) in which R⁸ is hydrogen may be prepared by reacting a compound of formula 5 with an isocyanate of formula O═C═N—R⁵.

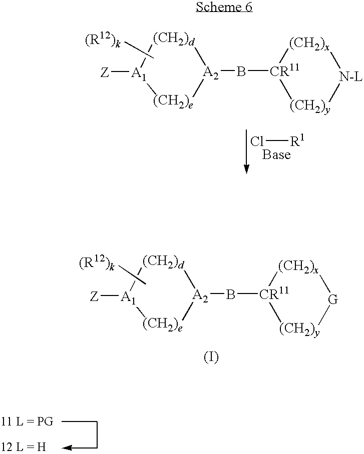

It will be appreciated that various functional group modifications may be made to compounds of formula (I) to form further compounds of formula (I) e.g. bearing different substituents on Z. Thus, for example, where Z is alkyl carboxyaryl further modification by hydrolysis and standard amide coupling may be carried out to give amide examples. Where Z is nitroaryl further modification may be carried out by hydrogenation with Pd on carbon catalysis to the aniline and further functionalisation by acids/acid chlorides, sulfonyl chlorides and isocyanates/carbamyl chlorides will give amide, sulfonamide and urea examples. Where Z is cyanoaryl further modification may be carried out by treatment with hydroxylamine to give the amidoxime which can be condensed with acids to give oxadiazole examples. Where Z is the methylthioaryl further modification may be carried out by oxidation of the sulfide to the sulfoxide and sulfone, N-oxides can be isolated as a by-product of the sulfone oxidation.

Other compounds of formula (I) may be prepared by methods analogous to those described above or in the examples, or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates, such as those defined above, may be of use in the synthesis of compounds of formula (I) and are therefore also included within the scope of the invention, for example compounds of formula 12:

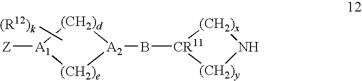

or a salt or protected derivative thereof, wherein the groups Z, A¹, A², B, R¹¹, R¹², d, e, k, x and y are as defined above for compounds of formula (I).

For compounds of formula 12:

i) when B represents an ethylene group; A₁ and A₂ each represent N; d, e, x, and y each represent 2; R¹¹ represents H; k represents 0; suitably Z does not represent:

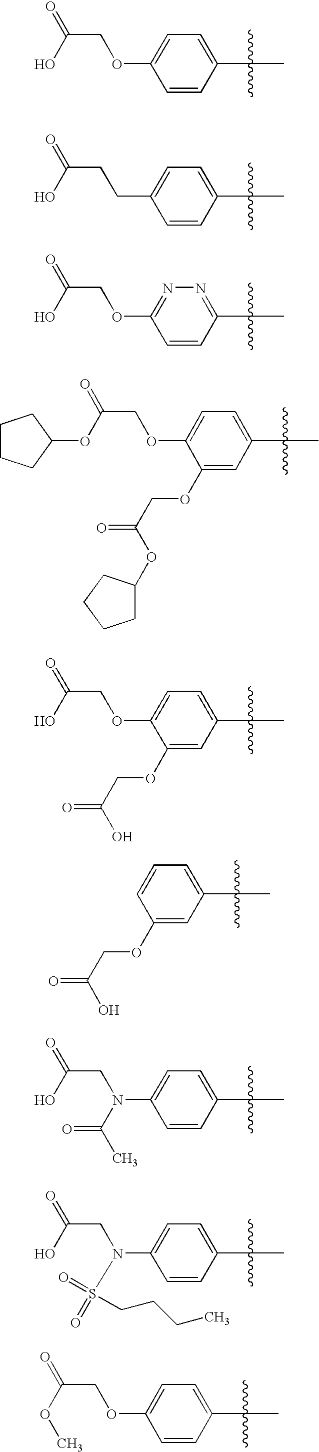

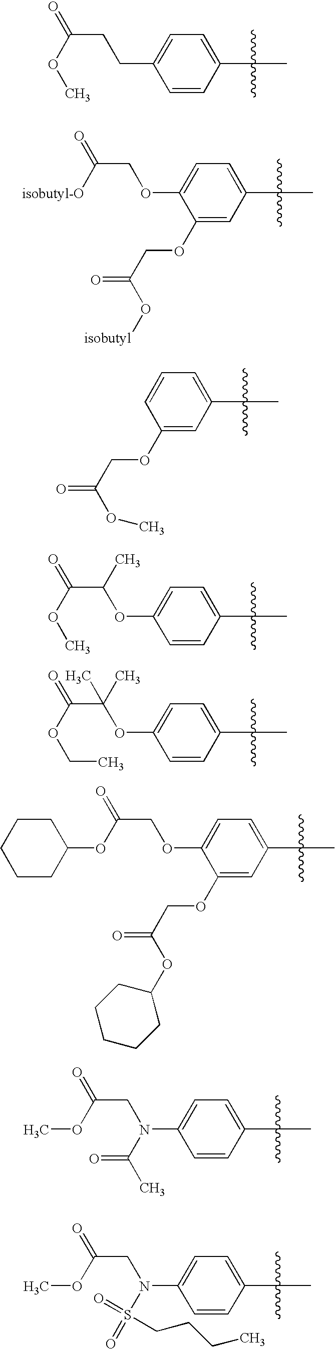

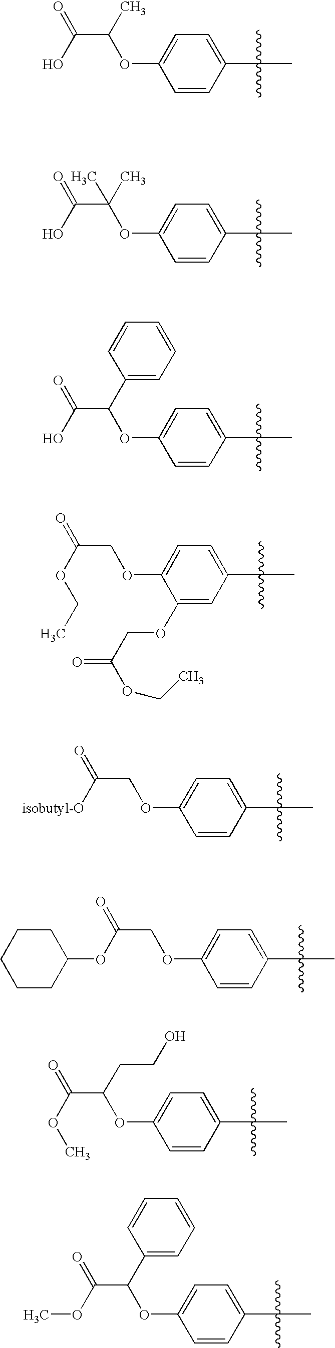

ii) when B represents a methylene group; A₁ and A₂ represent N; d and e each represent 2; x represents 1 and y represents 3, or x represents 2 and y represents 2; R¹¹ represents H; k represents 0; suitably Z does not represent 2-methoxyphenyl-.

iii) when B represents a methylene group; A₁ and A₂ represent N; d and e each represent 2; x represents 1 and y represents 3, or x represents 2 and y represents 2; R¹¹; represents H; k represents 0; suitably Z does not represent 1H-inod-4-yl-.

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The compounds of the invention may offer advantages over compounds acting via different mechanisms for the treatment of the above mentioned disorders in that they may offer beta-cell protection, increased cAMP and insulin secretion and also slow gastric emptying.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, dipeptidyl peptidase IV inhibitors, GLP-1 agonists e.g. GLP-1 analogues and mimetics, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution, containing 0.1% HCO₂H, over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes. Prep HPLC purification was carried out using a Lunar 10μ ODS2 (250×21.2 mm; Flow rate=20 mL/min) eluting with solvent A (0.05% TFA, 10% MeCN, 90% water) and solvent B (0.05% TFA, 90% MeCN, 10% water) and UV detection at 215 nm. Gradient information: 0.0-0.2 min: 90% A, 10% B; 0.2-10.0 min: Ramp up to 10% A, 90% B; 10.0-15.0 min: 10% A, 90% B; 15.0-16.0 min: Return to 90% A, 10% B.
Abbreviations and acronyms: Ac: Acetyl; tBDMS: tert-butyldimethylsilyl; Bn: Benzyl; t-Bu: tert-Butyl; Bz: Benzoyl; 18C6: [18]Crown-6; (Boc)₂O: Di-tert-butyl dicarbonate; DABCO: Bicyclo(2,2,2)-1,4-diazaoctane; DAST: Diethylammoniumsulfur trifloride: DBU: 1,8-Diazabicyclo[5.4.0]undec-7-ene; DIPEA: N,N-Diisopropylethylamine; DMAP: 4-Dimethylaminopyridine; DMF: N,N-Dimethylformamide; DMSO: Dimethylsulfoxide; EDCI: 1-(3-Dimethylaminopropyl)-3-ethylcarbodiimide hydrochloride; Et: Ethyl; i-Bu: Isobutyl; IH: Isohexane; i-Pr: Isopropyl; LiHMDS: Lithium bis(trimethylsilyl)amide; mCPBA: 3-Chloroperoxybenzoic acid; Me: Methyl; Ms: Methanesulfonyl; Ph: Phenyl; n-Pr: n-Propyl; RP-HPLC: Reverse phase-high performance liquid chromatography; rt: Room temperature; RT: Retention time; TFA: Trifluoroacetic acid; THF: Tetrahydrofuran; TMS: Trimethylsilyl. 4-Hydroxy-4-hydroxypropyl)piperidine-1-carboxylic acid tert-butyl ester: Cooper L. C., et al., Bioorg. Med. Chem. Lett., 2002, 12, 1759-1763; 4-(2-bromoacetyl)piperidine-1-carboxylic acid tert-butyl ester: WO2004/041777; 4-ethoxycarbonylmethylenepiperidine-1-carboxylic acid tert-butyl ester: Hetrocycles, 2001, 54, 2, 747-755; 1-(2-Bromoethyl)-4-methanesulfonylbenzene: WO199843956.

### Example 1

### 4-[4-(4-Methanesulfonylphenyl)piperazin-1-ylmethyl]piperidine-1-carboxylic Acid tert-butyl Ester

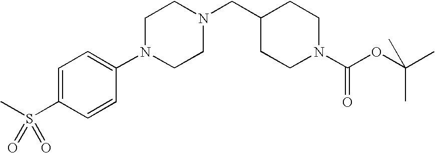

To a solution of 1-(4-methanesulfonylphenyl)piperazine (0.41 mmol) and 4-formylpiperidine-1-carboxylic acid tert-butyl ester (1.2 mmol) in DCM (3 mL) was added sodium triacetoxyborohydride (0.53 mmol). The resulting suspension was stirred at rt for 17 h. Polymer-supported isocyante scavenger resin (MP-NCO) (0.29 g, 1.44 mmol/g) was added and shaking continued until LCMS showed complete consumption of starting amine. The mixture was diluted with further DCM, shaken with water, and the organic layer separated using a hydrophobic frit. The crude mixture was purified via ion-exchange using an SCX column, to afford the title compound. δ_{H} (400 MHz, CHCl₃) 1.14 (2H, m), 1.50 (9H, s), 1.70 (1H, m), 1.79 (2H, m), 2.26 (2H, d), 2.58 (4H, t), 2.74 (2H, m), 3.04 (3H, s), 3.38 (4H, t), 4.14 (2H, m), 6.96 (2H, d), 7.80 (2H, d).

The compounds shown in Table 1 below were synthesised by analogous methods from the appropriate aldehyde and piperazine:

TABLE 1

| | | | RT | m/z |
|---|---|---|---|---|
| Eg | Structure | Name | (min) | (ES+) |
| 2 | 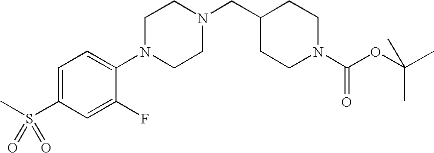 | 4-[4-(2-Fluoro-4-methane sulfonylphenyl)piperazin-1- ylmethyl]piperidine-1- carboxylic acid tert-butyl ester | 2.47 | 456.19 |
| 3 | 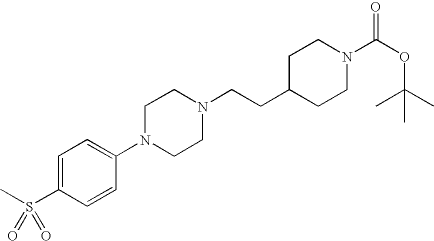 | 4-{2-[4-(4-Methanesulfonyl- phenyl)piperazin-1-yl]-ethyl}- piperidine-1-carboxylic acid tert-butyl ester | 2.64 | 452.29 |
| 4 | 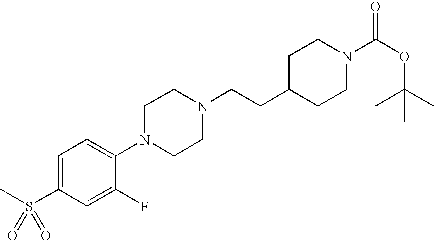 | 4-{2-[4-(2-Fluoro-4-methane sulfonylphenyl)piperazin-1- yl]ethyl}piperidine-1- carboxylic acid tert-butyl ester | 2.62 | 470.28 |
| 5 | 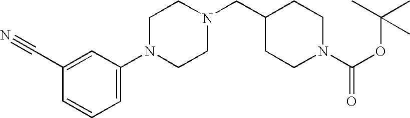 | 4-[4-(3-Cyanophenyl) piperazin-1-ylmethyl] piperidine-1-carboxylic acid tert-butyl ester | 2.79 | 385.28 |
| 6 | 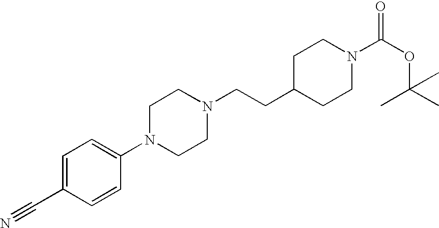 | 4-{2-[4-(4-Cyanophenyl) piperazin-1-yl]ethyl} piperidine-1-carboxylic acid tert-butyl ester | 2.77 | 399.28 |
| 7 | 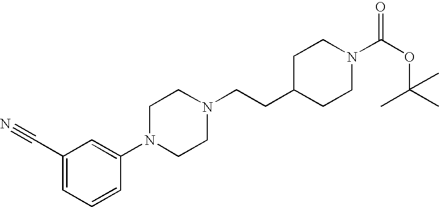 | 4-{2-[4-(3-Cyanophenyl) piperazin-1-yl]ethyl} piperidine-1-carboxylic acid tert-butyl ester | 2.72 | 399.25 |
| 8 | 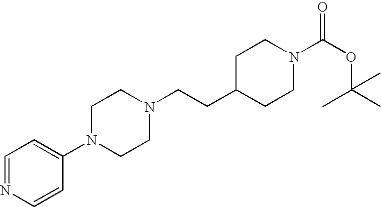 | 4-[2-(4-Pyridin-4-ylpiperazin- 1-yl)ethyl]piperidine-1- carboxylic acid tert-butyl ester | 2.07 | 375.20 |
| 9 | 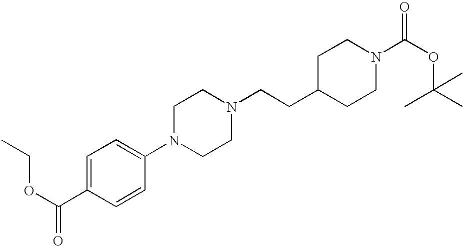 | 4-{2-[4-(4-Ethoxycarbonyl phenyl)piperazin-1-yl]ethyl} piperidine-1-carboxylic acid tert-butyl ester | 2.67 | 446.18 |
| 10 | 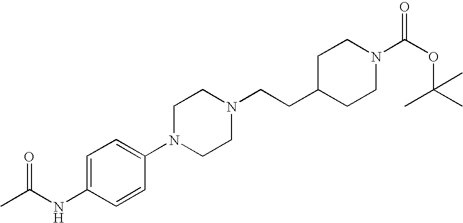 | 4-{2-[4-(4-Acetylamino phenyl)piperazin-1-yl]ethyl} piperidine-1-carboxylic acid tert-butyl ester | 2.51 | 431.33 |
| 11 | 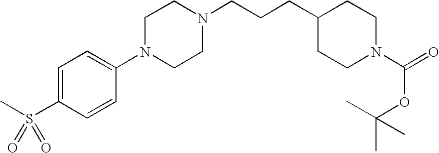 | 4-{3-[4-(4-Methanesulfonyl phenyl)piperazin-1-yl] propyl}piperidine-1- carboxylic acid tert-butyl ester | 2.61 | 466.42 |

### Example 12

### 4-{2-[4-(5-Fluoro-2-methanesulfonylphenyl)piperazin-1-yl]ethyl}piperidine-1-carboxylic acid tert-butyl ester

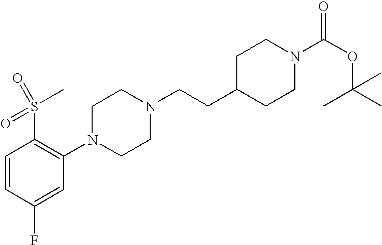

A solution of 2,4-difluorophenylmethylsulfone (0.10 g, 0.52 mmol) and piperazine (45 mg, 0.52 mmol) in tert-BuOH (2 mL) was stirred for 72 h at rt. The reaction mixture was diluted with MeOH and purified by ion-exchange chromatography (SCX) to give 1-(5-fluoro-2-methanesulfonylphenyl)piperazine. To a solution of 1-(5-fluoro-2-methane sulfonylphenyl)piperazine (75 mg, 0.25 mmol) and 4-(2-oxoethyl)piperidine-1-carboxylic acid tert-butyl ester (157 mg, 0.75 mmol) in DCM (2 mL) was added sodium triacetoxy borohydride (52 mg, 0.38 mmol) and the mixture was stirred at rt for 7 days. The reaction mixture was diluted with DCM, washed with water and purified by flash chromatography eluting with EtOAc to afford the title compound: RT=2.64 min; m/z (ES⁺)=470.14 [M+H]⁺.

### Example 13

### 4-{2-[4-(4-Carboxyphenyl)piperazin-1-yl]ethyl}piperidine-1-carboxylic Acid tert-butyl Ester

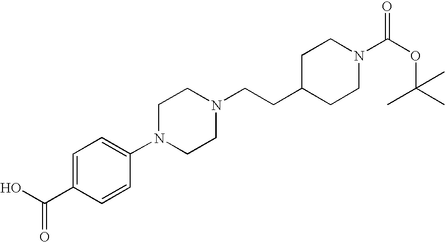

To a solution of 4-{2-[4-(4-ethoxycarbonylphenyl)piperazin-1-yl]ethyl}piperidine-1-carboxylic acid tert-butyl ester (3.73 g, 8.36 mmol) in MeOH (40 mL) was added 1 M NaOH (16.73 mL, 16.73 mmol) in water. The reaction was heated at 60° C. for 3 h, the mixture was cooled to rt and extracted with Et₂O. The aqueous phase was neutralised with 1 M HCl solution and extracted with EtOAc, the extracts were dried (MgSO₄) and the solvent was removed under vacuum to give the title compound: RT=2.49 min; m/z (ES⁺)=418.18 [M+H]⁺.

### Example 14

### 4-{2-[4-(4-Carbamoylphenyl)piperazin-1-yl]ethyl}piperidine-1-carboxylic Acid tert-butyl Ester

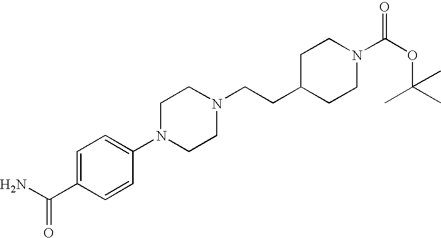

To a solution of 4-{2-[4-(4-carboxyphenyl)piperazin-1-yl]ethyl}piperidine-1-carboxylic acid tert-butyl ester (30 mg, 70 μmol), 0.5 M ammonia in dioxane (0.29 mL, 140 μmol) and Et₃N (15 μL, 110 μmol) in dimethylacetamide (0.3 mL) was added HBTU (41 mg, 110 μmol) in dimethylacetamide (0.3 mL) and the reaction was stirred for 20 h. The mixture was diluted with EtOAc washed with saturated NaC₂O₃ solution, dried (MgSO₄) and the solvent was removed under vacuum. The mixture was purified by chromatography on OPTIX 10 with a solvent gradient from 1:98:2 to 1:89:10 Et₃N: DCM: MeOH. The resulting mixture was taken up in DCM, washed with 1 M NaOH solution, dried (MgSO₄) and the solvent was removed under vacuum to afford the title compound: RT=2.49 min; m/z (ES⁺)=417.32 [M+H]⁺.

The compounds shown in Table 2 below were synthesised by analogous methods from 4-{2-[4-(4-carboxyphenyl)piperazin-1-yl]ethyl}piperidine-1-carboxylic acid tert-butyl ester and the appropriate amine:

TABLE 2

| | | | RT | m/z |
|---|---|---|---|---|
| Eg | Structure | Name | (min) | (ES+) |
| 15 | 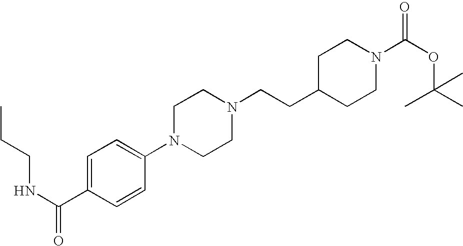 | 4-{2-[4-(4-Propylcarbamoyl phenyl)piperazin-1-yl]ethyl} piperidine-1-carboxylic acid tert-butyl ester | 2.79 | 459.39 |
| 16 | 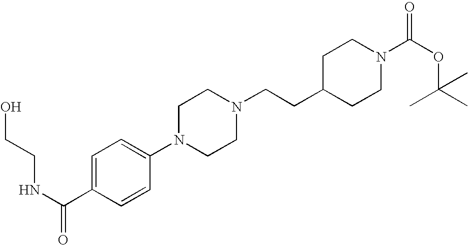 | 4-(2-{4-[4-(2-Hydroxyethyl carbamoyl)phenyl]piperazin-1- yl}ethyl)piperidine-1- carboxylic acid tert-butyl ester | 2.56 | 461.34 |
| 17 | 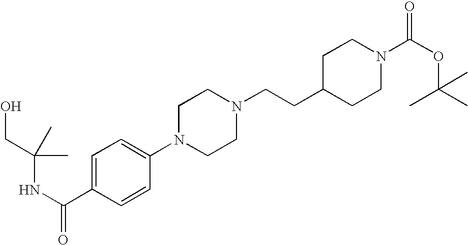 | 4-(2-{4-[4-(2-Hydroxy-1,1- dimethylethylcarbamoyl)phenyl] piperazin-1-yl}ethyl) piperidine-1-carboxylic acid tert-butyl ester | 2.67 | 489.36 |
| 18 | 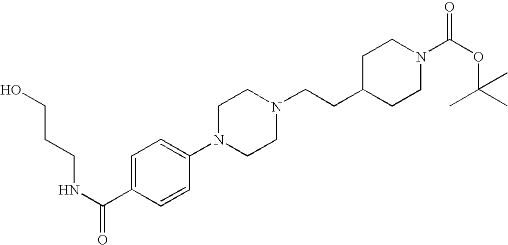 | 4-(2-{4-[4-(3-Hydroxypropyl carbamoyl)phenyl]piperidine-1- yl}ethyl)piperidine-1- carboxylic acid tert-butyl ester | 2.49 | 475.36 |
| 19 | 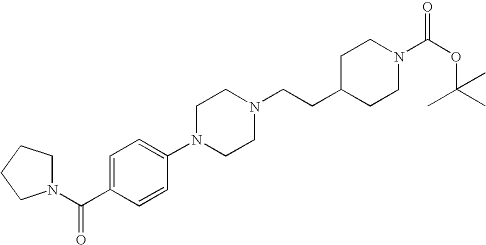 | 4-(2-{4-[4-(Pyrrolidine-1- carbonyl)phenyl]piperazin-1- yl}ethyl)piperidine-1- carboxylic acid tert-butyl ester | 2.62 | 471.35 |
| 20 | 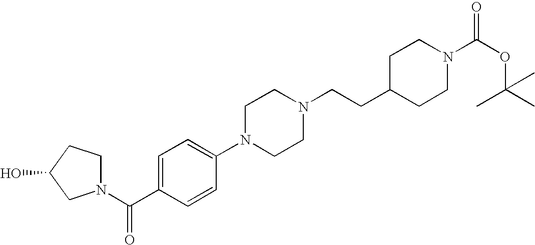 | 4-(2-{4-[4-((R)-3-Hydroxy pyrrolidine-1-carbonyl)phenyl] piperazin-1-yl}ethyl)piperidine- 1-carboxylic acid tert-butyl ester | 2.51 | 487.34 |
| 21 | 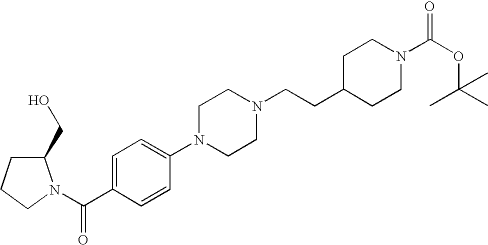 | 4-(2-{4-[4-((S)-2-Hydroxy methylpyrrolidine-1-carbonyl) phenyl]piperazin-1-yl}ethyl) piperidine-1-carboxylic acid tert-butyl ester | 2.59 | 501.35 |
| 22 | 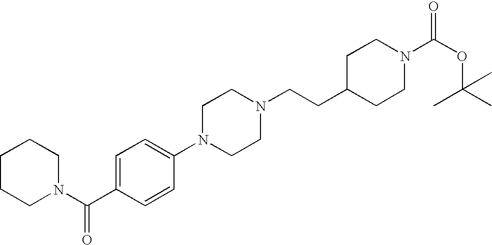 | 4-(2-{4-[4-(Piperidine-1- carbonyl)phenyl]piperazin-1- yl}ethyl)piperidine-1- carboxylic acid tert-butyl ester | 2.77 | 485.36 |
| 23 | 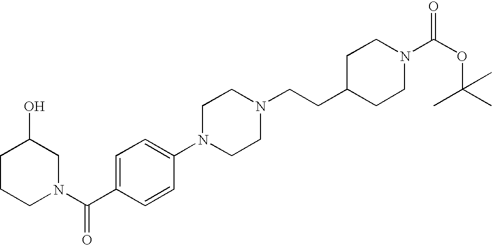 | 4-(2-{4-[4-(3-Hydroxy piperidine-1-carbonyl)phenyl] piperazin-1-yl}ethyl)piperidine- 1-carboxylic acid tert-butyl ester | 2.49 | 501.36 |
| 24 | 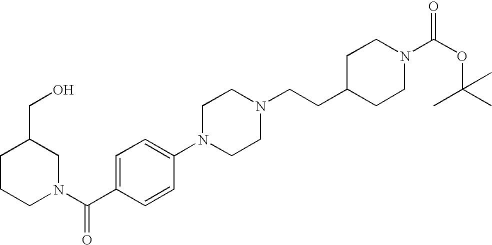 | 4-(2-{4-[4-(3-Hydroxymethyl- piperidine-1-carbonyl)phenyl] piperazin-1-yl}-ethyl) piperidine-1-carboxylic acid tert-butyl ester | 2.62 | 515.37 |
| 25 | 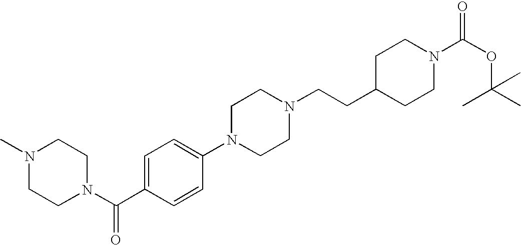 | 4-(2-{4-[4-(4-Methyl piperazine-1-carbonyl)phenyl] piperazin-1-yl}ethyl)piperidine- 1-carboxylic acid tert-butyl ester | 2.12 | 500.34 |

Intermediate 1: 2-[4-(4-Aminophenyl)piperazin-1-yl]ethyl}piperidine-1-carboxylic Acid tert-butyl Ester

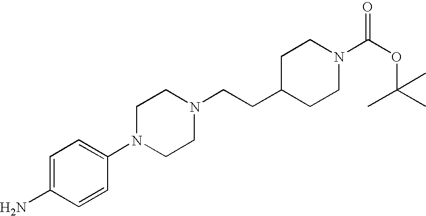

A solution of 4-(2-oxoethyl)piperidine-1-carboxylic acid tert-butyl ester (0.50 g, 2.20 mmol) and 1-(4-nitrophenyl)piperazine (0.35 g, 1.70 mmol) in anhydrous MeOH (5 mL) was stirred for 71 h at rt, then NaBH₄ (0.13 g, 3.39 mmol) was added and reaction was stirred for a further 3 h. The solvent was removed under vacuum and the resulting residue was partitioned between EtOAc and saturated NaHCO₃ solution. The aqueous phase was extracted with twice EtOAc, the organic extracts were combined, dried (MgSO₄) and adsorbed onto SiO₂. The adsorbed sample was purified by flash chromatography eluting with EtOAc to give 4-{2-[4-(4-nitrophenyl)piperazin-1-yl]ethyl}cyclohexanecarboxylic acid tert-butyl ester (0.54 g, 1.30 mmol), which was taken up in EtOH (25 mL), 10% palladium on carbon was added and the mixture was stirred under an hydrogen atmosphere at rt for 20 h. The reaction mixture was filtered through celite and the solvent was removed under vacuum to afford the title compound: δ_{H} (400 MHz, CHCl₃) 1.17 (2H, m), 1.48 (9H, s), 1.50 (1H, m), 1.69 (2H, d), 2.45 (2H, m), 2.62 (4H, br s), 2.71 (2H, m), 3.09 (4H, m), 3.44 (2H, br s), 4.09 (2H, br s), 6.68 (2H, d), 6.84 (2H, d).

### Example 26

### 4-{2-[4-(4-Propionylaminophenyl)piperazin-1-yl]ethyl}cyclo hexanecarboxylic acid tert-butyl ester

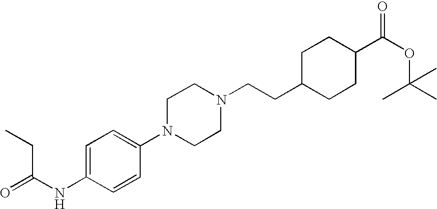

To a solution of 4-{2-[4-(4-aminophenyl)piperazin-1-yl]ethyl}cyclohexane carboxylic acid tert-butyl ester (40 mg, 0.10 mmol) and Et₃N (32 μL, 0.23 mmol) in DCM (3 mL) was added propionyl chloride (9.9 μL, 0.11 mmol) and the reaction was stirred for 72 h at rt. The reaction mixture was washed with saturated NaHCO₃ solution, adsorbed onto SiO₂ and purified by chromatography on OPTIX 10 eluting with 5:95 MeOH:DCM to afford the title compound: RT=2.44 min; m/z ES⁺)=445.39 [M+H]⁺.

The compounds shown in Table 3 below were synthesised by analogous methods from 4-{2-[4-(4-aminophenyl)piperazin-1-yl]ethyl}cyclohexane carboxylic acid tert-butyl ester and the appropriate acid chloride:

TABLE 3

| | | | RT | m/z |
|---|---|---|---|---|
| Eg | Structure | Name | (min) | (ES+) |
| 27 | 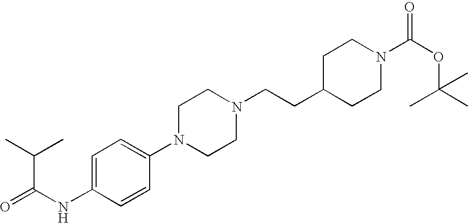 | 4-{2-[4-(4-Isobutyrylamino phenyl)piperazin-1-yl]ethyl} piperidine-1-carboxylic acid tert-butyl ester | 2.49 | 459.41 |
| 28 | 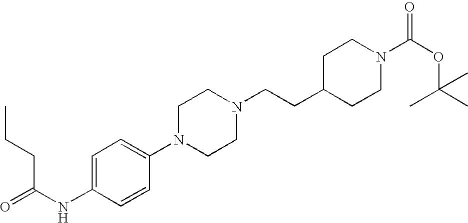 | 4-{2-[4-(4-Butyrylamino phenyl)piperazin-1-yl]ethyl} piperidine-1-carboxylic acid tert-butyl ester | 2.62 | 459.41 |
| 29 | 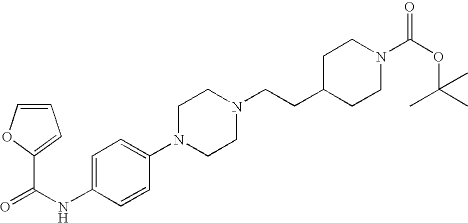 | 4-[2-(4-{4-[(Furan-2-carbonyl) amino]phenyl}piperazin-1-yl) ethyl]piperidine-1-carboxylic acid tert-butyl ester | 2.61 | 483.38 |
| 30 | 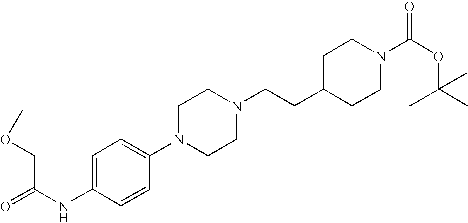 | 4-(2-{4-[4-(2-Methoxyacetyl amino)phenyl]piperazin-1-yl} ethyl)piperidine-1-carboxylic acid tert-butyl ester | 2.47 | 461.39 |
| 31 | 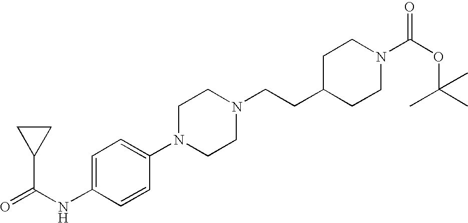 | 4-(2-{4-[4-(Cyclopropane carbonylamino)phenyl] piperazin-1-yl}ethyl)piperidine- 1-carboxylic acid tert-butyl ester | 2.67 | 457.40 |

### Example 32

### 4-[2-(4-{4-[2-(2-Methoxyethoxy)acetylamino]phenyl}piperazin-1-yl)ethyl]piperidine-1-carboxylic acid tert-butyl ester

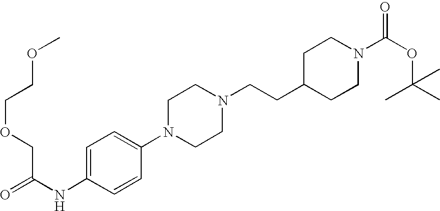

A solution of 4-{2-[4-(4-aminophenyl)piperazin-1-yl]ethyl}cyclohexane carboxylic acid tert-butyl ester (40 mg, 0.10 mmol), (2-methoxyethoxy)acetic acid (14 mg, 0.10 mmol), DIPEA (44 mg, 0.34 mmol) and HOBT.H₂O (17.4 mg, 0.11 mmol) in DMF (3 mL) was stirred for 10 min and EDCI (24 mg, 0.12 mmol) was added then the mixture was stirred for 24 h. The solvent was removed under vacuum and the resulting residue was partitioned between saturated NaHCO₃ solution and DCM. The organic phase was collected and adsorbed onto SiO₂ then purified by chromatography on OPTIX 10 eluting with 5:95 MeOH:DCM to afford the title compound. RT=2.59 min; m/z (ES⁺)=505.41 [M+H]⁺.

The compounds shown in Table 4 below were synthesised by analogous methods from 4-{2-[4-(4-aminophenyl)piperazin-1-yl]ethyl}cyclohexane carboxylic acid tert-butyl ester and the appropriate carboxylic acid:

TABLE 4

| | | | RT | m/z |
|---|---|---|---|---|
| Eg | Structure | Name | (min) | (ES+) |
| 33 | 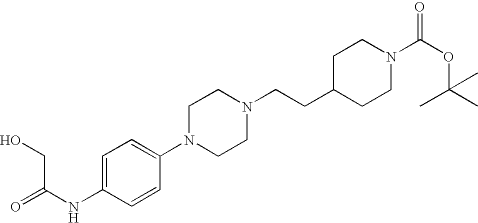 | 4-(2-{4-[4-(2-Hydroxyacetyl amino)phenyl]piperazin-1-yl} ethyl)piperidine-1-carboxylic acid tert-butyl ester | 2.39 | 447.36 |
| 34 | 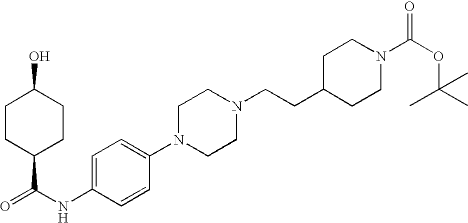 | 4-[2-(4-{4-[(4-Hydroxycyclo hexanecarbonyl)amino]phenyl piperazin-1-yl)ethyl]piperidine- 1-carboxylic acid tert-butyl ester | 2.54 | 515.43 |
| 35 | 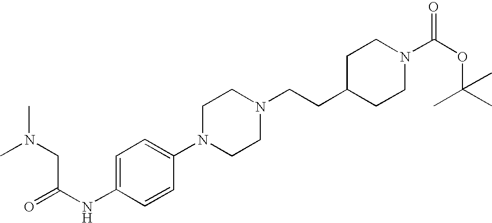 | 4-(2-{4-[4-(2-Dimethylamino acetylamino)phenyl]piperazin- 1-yl}ethyl)piperidine-1- carboxylic acid tert-butyl ester | 2.09 | 474.37 |
| 36 | 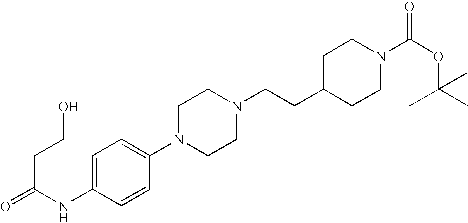 | 4-(2-{4-[4-(3-Hydroxy propionylamino)phenyl] piperazin-1-yl}ethyl)piperidine- 1-carboxylic acid tert-butyl ester | 2.40 | 461.40 |

Intermediate 2: 4-(2-{4-[4-N-Hydroxycarbamimidoyl)phenyl]piperazin-1-yl}ethyl)piperidine-1-carboxylic acid tert-butyl ester

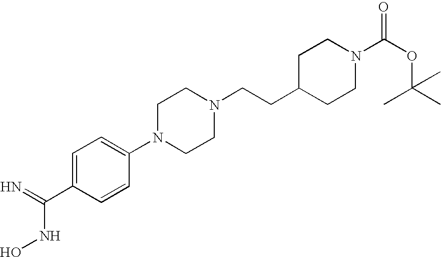

To a solution of 4-{2-[4-(4-cyanophenyl)piperazin-1-yl]ethyl}piperidin-1-carboxylic acid tert-butyl ester (1.23 g, 3.08 mmol) in EtOH (20 mL) was added K₂CO₃ (0.85 g, 6.16 mmol) followed by a solution of hydroxylamine hydrochloride (0.43 g, 6.20 mmol) in water. The reaction was heated at 85° C. for 24 h, the mixture was then partitioned between water and EtOAc. The aqueous phase was re-extracted with EtOAc, the organic extracts were combined, washed with brine, dried (MgSO₄) and adsorbed onto SiO₂. The adsorbed sample was purified by flash chromatography eluting with 10:90 MeOH:DCM to afford the title compound: δ_{H} (400 MHz, CHCl₃) 1.16 (2H, m), 1.48 (9H, s), 1.50 (1H, m), 1.70 (2H, d), 2.46 (2H, m), 2.61 (4H, br s), 2.71 (2H, m), 3.28 (4H, m), 4.82 (1H, s), 5.32 (2H, s), 6.92 (2H, d), 7.54 (2H, d).

### Example 37

### 4-(2-{4-[4-(5-Methyl[1,2,4]oxadiazol-3-yl)phenyl]piperazin-1-yl}ethyl)piperidine-1-carboxylic acid tert-butyl ester

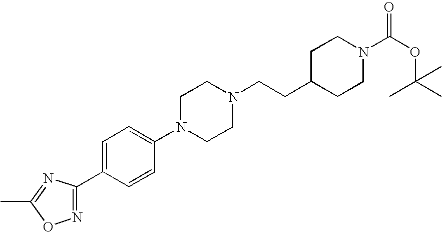

To a solution of 4-(2-{4-[4-(N-hydroxycarbamimidoyl)phenyl]piperazin-1-yl}ethyl)piperidine-1-carboxylic acid tert-butyl ester (26 mg, 60 μmol), AcOH (3 μL, 55 μmol) and HOBT.H₂O (9.2 mg, 60 μmol) in DMF (2 mL) was added EDCI (12.6 mg, 66 μmol) and the mixture was stirred for 10 min at rt. The solvent was removed under vacuum and the resulting residue was partitioned between saturated NaHCO₃ solution and EtOAc. The aqueous phase was re-extracted with EtOAc, the organic extracts were combined, washed with brine, dried (MgSO₄) and the solvent was removed under vacuum. The residue was taken up in toluene and refluxed for 6 h. The reaction mixture was adsorbed onto SiO₂ and purified by flash chromatography eluting with 3:97 MeOH:DCM to afford the title compound: RT=2.65 min; m/z (ES⁺)=456.33 [M+H]⁺.

The compounds shown in Table 5 below were synthesised by analogous methods from 4-(2-{4-[4-(N-hydroxycarbamimidoyl)phenyl]piperazin-1-yl}ethyl)piperidine-1-carboxylic acid tert-butyl ester and the appropriate carboxylic acid:

TABLE 5

| | | | RT | m/z |
|---|---|---|---|---|
| Eg | Structure | Name | (min) | (ES+) |
| 38 | 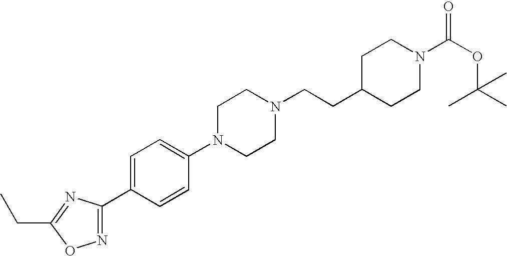 | 4-(2-{4-[4-(5-Ethyl-[1,2,4] oxadiazol-3-yl)phenyl] piperazin-1-yl}ethyl) piperidine-1-carboxylic acid tert-butyl ester | 3.20 | 470.39 |
| 39 | 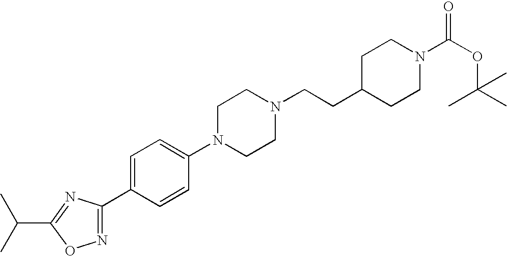 | 4-(2-{4-[4-(5-Isopropyl-[1,2,4] oxadiazol-3-yl)phenyl] piperazin-1-yl}ethyl) piperidine-1-carboxylic acid tert-butyl ester | 3.01 | 484.36 |

### Example 40

### 4-(2-{4-[4-(3-Isopropyl[1,2,4]oxadiazol-5-yl)phenyl]piperazin-1-yl}ethyl)piperidine-1-carboxylic acid tert-butyl ester

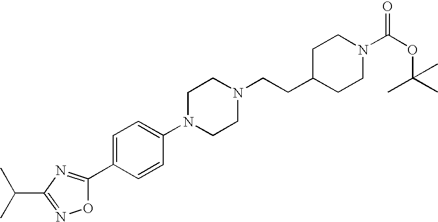

The title compound was prepared using the same procedure used to synthesise 4-(2-{4-[4-(5-methyl[1,2,4]oxadiazol-3-yl)phenyl]piperazin-1-yl}ethyl)piperidine-1-carboxylic acid tert-butyl ester from 4-{2-[4-(4-carboxyphenyl)piperazin-1-yl]ethyl}piperidine-1-carboxylic acid tert-butyl ester and N-hydroxyisobutyramidine: RT=3.01 min; m/z (ES⁺)=484.40 [M+H]⁺.

### Example 41

### 4-(2-{4-[4-(3-Isopropyl[1,2,4]oxadiazol-5-yl)phenyl]piperazin-1-yl}ethyl)piperidine-1-carboxylic acid tert-butyl ester

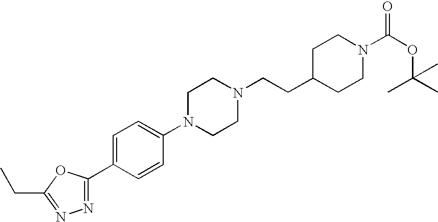

4-{2-[4-(4-Ethoxycarbonylphenyl)piperazin-1-yl]ethyl}piperidine-1-carboxylic acid tert-butyl ester (316 mg, 0.71 mmol) and hydrazine hydrate (0.44 mL, 7.10 mmol) in EtOH (10 mL) were refluxed for 88 h. The solvent was removed by evaporation and the resulting solid triturated (EtOAc) to give 4-{2-[4-(4-hydrazinocarbonyl phenyl)piperazin-1-yl]ethyl}piperidine-1-carboxylic acid tert-butyl ester: RT=2.31 min; m/z (ES⁺)=432.34 [M+H]⁺. To a solution of 4-{2-[4-(4-hydrazinocarbonyl phenyl)piperazin-1-yl]ethyl}piperidine-1-carboxylic acid tert-butyl ester (16 mg, 37 μmol) and DIPEA (14.2 μL, 82 μmol) in THF (2 mL) was added propionyl chloride (4 μL, 41 μmol) and the reaction was stirred at rt for 20 h. Another batch of propionyl chloride (4 μL, 41 μmol) was added and the mixture was stirred for a further 24 h. The reaction mixture was partitioned between saturated NaHCO₃ solution and EtOAc. The aqueous phase was re-extracted with EtOAc, the organic extracts were combined, washed with brine, dried (MgSO₄) and adsorbed onto SiO₂. The adsorbed sample was purified by flash chromatography eluting with 5:95 MeOH:DCM to give 4-(2-{4-[4-(N′-acetylhydrazino carbonyl)phenyl]piperazin-1-yl}ethyl)piperidine-1-carboxylic acid tert-butyl ester. To a solution of 4-(2-{4-[4-(N′-acetylhydrazinocarbonyl)phenyl]piperazin-1-yl}ethyl)piperidine-1-carboxylic acid tert-butyl ester (17 mg, 35 μmol) and DIPEA (18 μL, 105 μmol) in DCM (3 mL) was added POCl₃ (4 μL, 38 μmol) and the mixture was stirred for 5 h. The reaction mixture was quenched with saturated NaHCO₃ solution. The mixture was diluted with DCM and the organic phase was collected. The aqueous phase was re-extracted with DCM, the organic extracts were combined, dried (MgSO₄) and adsorbed onto SiO₂. The adsorbed sample was purified by flash chromatography eluting with 5:95 MeOH:DCM to afford the title compound: RT=2.72 min; m/z (ES⁺)=470.25 [M+H]⁺.

### Example 42

### 4-[1-(4-Methanesulfonylphenyl)piperidin-4-yloxymethyl]piperidine-1-carboxylic acid tert-butyl ester

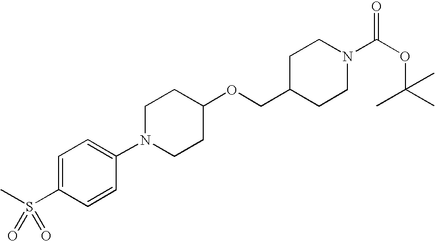

To a solution of 1-(4-methanesulfonylphenyl)piperidin-4-ol (0.10 g, 0.39 mmol) and 15-crown-5 (87 mg, 0.39 mmol) in anhydrous THF (3 mL) at 0° C. under argon was added a 60% dispersion of NaH in mineral oil (16 mg, 0.39 mmol) and the mixture was stirred for 30 min. 4-Methanesulfonyloxymethylpiperidine-1-carboxylic acid tert-butyl ester (0.23 g, 0.78 mmol) was added to the reaction and the mixture was heated by microwave irradiation to 100° C. for 30 min. The reaction was quenched with saturated NH₄Cl and extracted with EtOAc. The organic extracts were dried (MgSO₄), solvent was removed under vacuum and the resulting residue was purified by flash chromatography eluting with 1:1 EtOAc:hexane to afford the title compound: RT=3.81 min; m/z (ES⁺)=453.31 [M+H]⁺.

### Example 43

### 4-{[1-(4-Methanesulfonylphenyl)piperidin-4-ylamino]methyl}piperidine-1-carboxylic acid tert-butyl ester

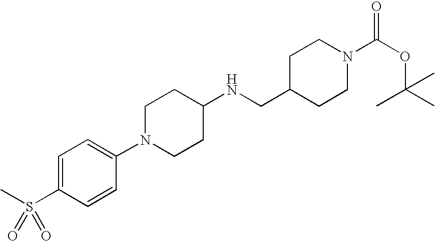

To a solution of 1-(4-methanesulfonylphenyl)piperidin-4-ol (0.89 g, 3.50 mmol) in DCM (25 mL) at 10° C. was added Dess-Martin periodinane (1.60 g, 3.77 mmol) and the reaction was stirred for 2 h. The reaction mixture was diluted with DCM, washed with 1 M NaOH solution, then brine, dried (MgSO₄), and the solvent was removed under vacuum to give 4-oxopiperidine-1-carboxylic acid tert-butyl ester. A mixture of 4-oxopiperidine-1-carboxylic acid tert-butyl ester (0.51 g, 2.40 mmol) and 4-aminomethyl piperidine-1-carboxylic acid tert-butyl ester (0.43 g, 2.01 mmol) in DCM (30 mL) was stirred for 30 min, then sodium triacetoxyborohydride (0.51 g, 2.41 mmol) was added and the mixture was stirred for 48 h. The reaction was diluted with DCM then washed with saturated NaHCO₃ solution then brine, dried (MgSO₄) and the solvent was removed under vacuum to give a residue which was purified by flash chromatography eluting with 10:90 MeOH:DCM to afford the title compound: RT=2.49 min; m/z (ES⁺)=452.25 [M+H]⁺.

### Example 44

### 4-{2-[4-(4-Sulfamoylphenyl)piperazin-1-yl]ethyl}piperidine-1-carboxylic Acid Tert-Butyl Ester Hydrochloride

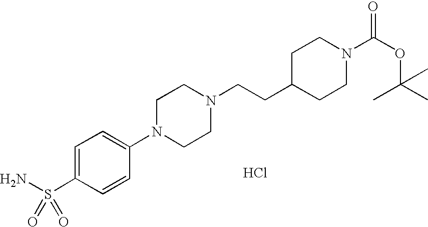

A mixture of 4-fluorobenzenesulfonamide (1.00 g, 5.71 mmol) and piperazine (2.46 g, 28.54 mmol) in water (12 mL) was heated at 100° C. for 20 h. The resulting precipitate was collected filtration and washed with water and toluene to give 4-piperazin-1-ylbenzenesulfonamide: RT=0.49 min; m/z (ES⁺)=242.13 [M+H]⁺. A solution of 4-piperazin-1-ylbenzenesulfonamide (0.46 g, 1.89 mmol) and 4-(2-oxoethyl)piperidine-1-carboxylic acid tert-butyl ester (0.43 g, 1.89 mmol) in DCM (50 mL) and THF (7 mL) with molecular sieves (0.90 g) was stirred under argon at rt for 1 h. Sodium acetoxyborohydride (0.52 g, 2.46 mmol) was added and the reaction mixture was stirred for a further 2.5 h. The reaction mixture was quenched with saturated NaHCO₃ solution and extracted with EtOAc. The organic extracts were washed with brine, dried (MgSO₄) and the solvent was removed under vacuum. The resulting solid was purified by recrystallisation (EtOAc) then dissolved in THF and 1 M HCl in dioxane (0.95 equivalents), the solvent was removed under vacuum and the resulting solid was washed with Et₂O to afford the title compound: RT=2.51 min; m/z (ES⁺)=453.33 [M+H]⁺.

### Example 45

### 4-{2-[4-(3-Fluoro-4-sulfamoylphenyl)piperazin-1-yl]ethyl}piperidine-1-carboxylic acid tert-butyl ester

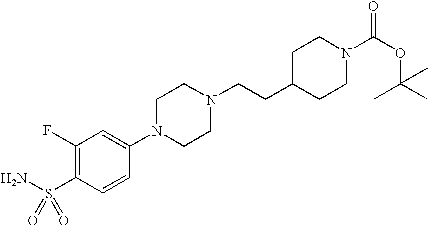

The same procedure was used that was used to synthesise 4-{2-[4-(4-sulfamoylphenyl)piperazin-1-yl]ethyl}piperidine-1-carboxylic acid tert-butyl. Purification was carried out by Prep HPLC to afford the title compound. RT=2.59 min; m/z (ES⁺)=471.34 [M+H]⁺.

### Example 46

### 4-{2-[4-(4-(Pyrrolidine-1-sulfonyl)phenyl)piperazin-1-yl]ethyl}piperidine-1-carboxylic acid tert-butyl ester

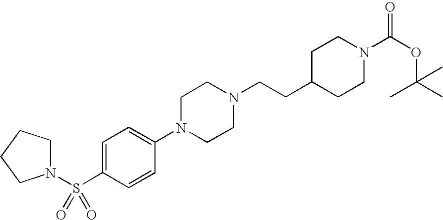

To a solution of pyrrolidine (95 μL, 1.13 mmol) and Et₃N (158 μL, 1.13 mmol) in DCM (2.5 mL) was added 4-fluorobenzenesulfonyl chloride (200 mg, 1.03 mmol) and the reaction was stirred at rt for 2 h. The reaction mixture was diluted with DCM, then washed with water then brine, dried (MgSO₄) and the solvent was removed under vacuum to give 1-(4-fluorobenzenesulfonyl)pyrrolidine: RT=3.06 min; m/z (ES⁺)=230.13 [M+H]⁺. A mixture of piperazine (47 mg, 0.55 mmol) and 1-(4-fluorobenzenesulfonyl)pyrrolidine (25 mg, 0.11 mg) in water (3 mL) was heated in a microwave at 150° C. for 30 min. The resulting solid was collected by filtration, washed with water and toluene to give 1-[4-(pyrrolidine-1-sulfonyl)phenyl]piperazine: RT=2.01 min; m/z (ES⁺)=296.15 [M+H]⁺. A solution of 1-[4-(pyrrolidine-1-sulfonyl)phenyl]piperazine (24 mg, 80 μmol) and 4-(2-oxoethyl)piperidine-1-carboxylic acid tert-butyl ester (18 mg, 80 μmol) in DCM (5 mL) with molecular sieves 50 mg) was stirred under argon at rt for 1 h. Sodium acetoxyborohydride (22 mg, 104 μmol) was added and the reaction mixture was stirred for a further 2.5 h. The reaction mixture was quenched with saturated NaHCO₃ solution and extracted with EtOAc. The organic extracts were washed with brine, dried (MgSO₄) and the solvent was removed under vacuum. The resulting solid was purified by flash chromatography eluting with 5:95 MeOH:DCM to afford the title compound: RT=2.69 min; m/z (ES⁺)=507.33 [M+H]⁺.

The compounds shown in Table 6 below were synthesised by analogous methods from 4-fluorobenzenesulfonyl chloride and the appropriate amine:

TABLE 6

| | | | RT | m/z |
|---|---|---|---|---|
| Eg | Structure | Name | (min) | (ES+) |
| 47 | 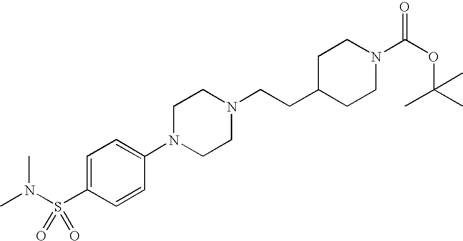 | 4-{2-[4-(4-Dimethylsulfamoyl phenyl)piperazin-1-yl]ethyl} piperidine-1-carboxylic acid tert-butyl ester | 2.69 | 481.43 |
| 48 | 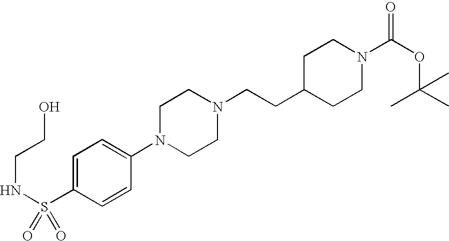 | 4-(2-{4-[4-(2-Hydroxyethyl sulfamoyl)phenyl]piperazin-1- yl}ethyl)piperidine-1- carboxylic acid tert-butyl ester | 2.36 | 497.30 |
| 49 | 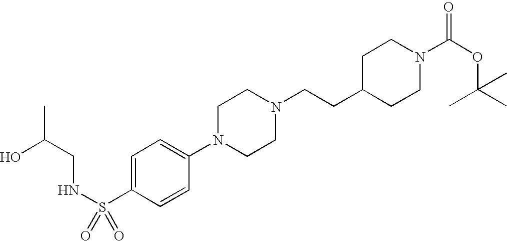 | 4-(2-{4-[4-(2-Hydroxypropyl sulfamoyl)phenyl]piperazin-1- yl}ethyl)piperidine-1- carboxylic acid tert-butyl ester | 2.43 | 511.31 |
| 50 | 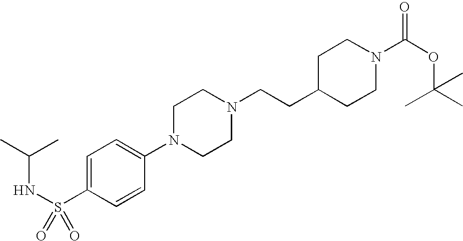 | 4-{2-[4-(4-Isopropylsulfamoyl phenyl)piperazin-1-yl]ethyl} piperidine-1-carboxylic acid tert-butyl ester | 2.61 | 495.22 |

### Intermediate 3: 1-(4-Methanesulfinylphenyl)piperazine

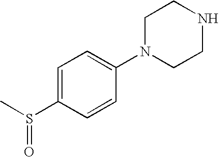

To a solution of 4-fluorothioanisole (2.0 g, 14.1 mmol) in DCM (10 mL) was added 60% mCPBA (4.06 g, 14.08 mmol) and the mixture was stirred overnight at rt. The reaction mixture was washed with 2 M NaOH solution, dried (MgSO₄) and purified by flash chromatography eluting with 40:60 EtOAc:hexane to give 1-fluoro-4-methanesulfinyl benzene. A mixture of 1-fluoro-4-methanesulfinylbenzene (0.75 g, 4.75 mmol) and piperazine (2.04 g, 23.7 mmol) in water (5 mL) was heated at 100° C. for 20 h. The reaction mixture was adsorbed onto SiO₂ and purified by flash chromatography eluting with 1:3:96 NH₃:MeOH:DCM to afford the title compound: RT=1.30 min; m/z (ES⁺)=225.10 [M+H]⁺.

### Example 51

### 4-{2-[4-(3-Fluoro-4-methanesulfonylphenyl)piperazin-1-yl]ethyl}piperidine-1-carboxylic acid tert-butyl ester

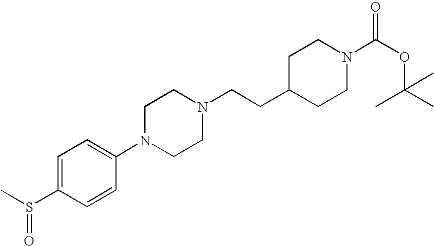

A solution of 1-(4-methanesulfinylphenyl)piperazine (46 mg, 0.22 mmol) and 4-(2-oxoethyl)piperidine-1-carboxylic acid tert-butyl ester (50 mg, 0.22 mmol) in anhydrous MeOH (2 mL) with glacial AcOH (1 drop) was stirred at rt under argon for 20 h. NaBH₄ (17 mg, 0.44 mmol) was added to the mixture and the reaction was stirred for a further 3 h. The reaction was quenched with water and extracted with DCM. The organic phase was collected and purified by flash chromatography eluting with 1:4:95 NH₃:MeOH:DCM to afford the title compound: RT=2.54 min; m/z (ES⁺)=436.33 [M+H]⁺.

Intermediate 4: 1-(3-Fluoro-4-methylsulfanylphenyl)piperazine

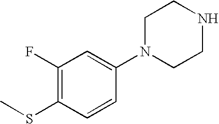

A mixture of bis(2-chloroethyl)amine (0.57 g, 3.18 mmol) and 3-fluoro-4-methylsulfanyl aniline (0.50 g, 3.18 mmol) in chlorobenzene (3 mL) was heated at 130° C. for 48 h. The reaction mixture was partitioned between DCM and saturated Na₂HCO₃ solution, the aqueous phase was then re-extracted with DCM. The organic extracts were combined, dried (MgSO₄) and the solvent was removed phase under vacuum. The mixture was purified by flash chromatography eluting with 10:90 MeOH:DCM to afford the title compound: RT=2.12 min; m/z (ES⁺)=227.07 [M+H]⁺.

### Example 52

### 4-({2-[4-(3-Fluoro-4-methanesulfonylphenyl)piperazin-1-yl]ethyl}-piperidine-1-carboxylic acid tert-butyl ester

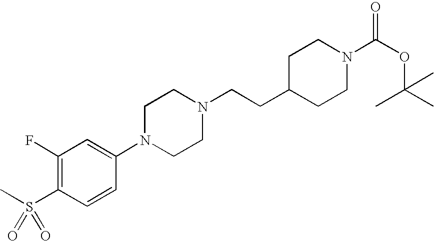

A solution of 1-(3-fluoro-4-methylsulfanylphenyl)piperazine (183 mg, 0.81 mmol) and 4-(2-oxoethyl)piperidin 1-carboxylic acid tert-butyl ester (368 mg, 1.62 mmol) in anhydrous MeOH (5 mL) with glacial AcOH (1 drop) was stirred at rt under argon for 20 h. NaBH₄ (92 mg, 2.43 mmol) was added to the mixture and the reaction was stirred for a further 3 h. The reaction was quenched with saturated Na₂HCO₃ solution and extracted with DCM. The organic phase was collected, washed with brine, dried (MgSO₄), the solvent was removed under vacuum and the resulting solid was purified by flash chromatography eluting with 50:50 EtOAc:hexane to give 4-{2-[4-(3-fluoro-4-methylsulfanylphenyl)piperazin-1-yl]ethyl}piperidine-1-carboxylic acid tert-butyl ester: RT=2.84 min; m/z (ES⁺)=438.30 [M+H]⁺. To a solution of 4-{2-[4-(3-fluoro-4-methylsulfanylphenyl)piperazin-1-yl]ethyl}piperidine-1-carboxylic acid tert-butyl ester (64 mg, 0.15 mmol), NaMoO₄ (3.5 mg, 15 μmol) and tributylamine (3.5 μL, 15 μmol) in toluene (1 mL) was added 27% H₂O₂ solution (10 μL, 79 mmol) followed by glacial AcOH (47.5 μL, 0.80 mmol) and finally 27% H₂O₂ solution (27 μL, 211 μmol). The reaction was warmed to 60° C. for 30 min, then quenched with 10% Na₂SO₃ solution and the aqueous phase was basified to pH8 with 1 M NaOH solution. The mixture was extracted with EtOAc, the organic phase was dried (MgSO₄), solvent was removed under vacuum and the resulting residue was purified by flash chromatography eluting with EtOAc then 10:90 MeOH:DCM to afford the title compound: RT=2.57 min; m/z (ES⁺)=470.35 [M+H]⁺.

### Example 53

### 4-{2-[4-(3-Fluoro-4-methanesulfonylphenyl)-1-oxypiperazin-1-yl]ethyl}piperidine-1-carboxylic acid tert-butyl ester

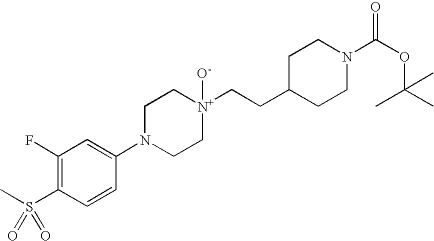

4-{2-[4-(3-Fluoro-4-methanesulfonylphenyl)-1-oxypiperazin-1-yl]ethyl}piperidine-1-carboxylic acid tert-butyl ester was prepared in the above reaction and isolated by flash chromatography eluting with 10:90 MeOH:DCM to afford the title compound. RT=2.70 min; m/z (ES⁺)=486.27 [M+H]⁺.

### Intermediate 5: 1-(4-Ethylsulfanylphenyl)piperazine

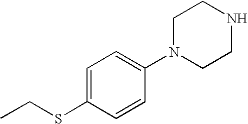

Argon was bubbled through a solution of 4-aminothiophenol (1.0 g, 8.00 mmol) in EtOH (10 mL) for 5 min. Ethyl iodide (1.37 g, 8.80 mmol) was added to the reaction followed by NaOMe (0.43 g, 8.00 mmol) and the mixture was heated at 70° C. under argon for 18 h. The solvent was removed under vacuum and the resulting residue was purified by Prep HPLC to give 4-ethylsulfanylaniline: RT=1.71 min; m/z (ES⁺)=154.08 [M+H]⁺. A mixture of bis(2-chloroethyl)amine (0.21 g, 1.20 mmol) and 4-ethylsulfanylaniline (0.19 g, 1.14 mmol) in chlorobenzene (2 mL) was heated at 130° C. for 48 h. The reaction mixture was partitioned between EtOAc and 2 M NaOH solution, then the solvent was removed from the organic phase under vacuum. The mixture was purified by flash chromatography eluting with 1:3:96 NH₃:MeOH:DCM to afford the title compound: RT=2.27 min; m/z (ES⁺)=223.12 [M+H]⁺.

### Example 54

### 4-{2-[4-(4-Ethanesulfonylphenyl)piperazin-1-yl]ethyl}piperidine-1-carboxylic acid tert-butyl ester

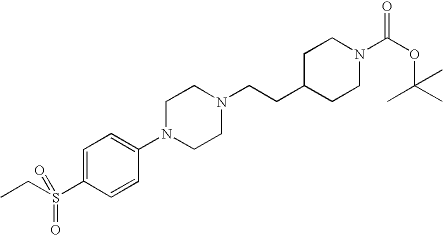

A solution of 1-(4-ethylsulfanylphenyl)piperazine (80 mg, 0.36 mmol) and 4-(2-oxoethyl)piperidine-1-carboxylic acid tert-butyl ester (82 mg, 0.36 mmol) in anhydrous MeOH (2 mL) with glacial AcOH (1 drop) was stirred at rt under argon for 20 h. NaBH₄ (27 mg, 0.72 mmol) was added to the mixture and the reaction was stirred for a further 3 h. The reaction was quenched with water and extracted with DCM. The organic phase was collected, dried (MgSO₄), the solvent was removed under vacuum and the resulting solid was purified by flash chromatography eluting with 1:2:97 NH₃:MeOH:DCM to give 4-{2-[4-(4-ethyl sulfanylphenyl)piperazin-1-yl]ethyl}piperidine-1-carboxylic acid tert-butyl ester: RT=3.14 min; m/z (ES⁺)=448.36 [M+H]⁺. To a solution of 4-{2-[4-(4-ethylsulfanylphenyl)piperazin-1-yl]ethyl}piperidine-1-carboxylic acid tert-butyl ester (90 mg, 208 μmol), NaMoO₄ (5 mg, 20.8 μmol) and tributylamine (5 μL, 20.8 μmol) in toluene (1 mL) was added 27% H₂O₂ solution (20 μL, 160 μmol) followed by glacial AcOH (13 μL, 229 μmol) and finally 27% H₂O₂ solution (32 μL, 256 μmol). The reaction was quenched after 10 min with 10% Na₂SO₃ solution and extracted with DCM. The organic phase was dried (MgSO₄) and purified by flash chromatography eluting with 1:2:97 NH₃:MeOH:DCM to afford the title compound: RT=2.61 min; m/z (ES⁺)=466.25 [M+H]⁺.

Intermediate 6: 4-(4-Methylsulfanylphenyl)piperidine-1-carboxylic acid tert-butyl ester

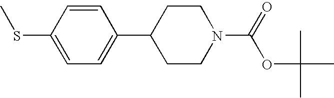

To a solution of 4-(4-methylsulfanylphenyl)piperidine hydrochloride (0.5 g, 2.05 mmol) in dioxane (10 mL) was added (Boc)₂O (0.47 g, 2.15 mmol) followed by water (2.5 mL) at rt. The resulting mixture was allowed to stir for 1 h. The solvent was removed in vacuo and the crude material diluted with EtOAc (75 mL) and water (25 mL). The two layers were separated and the aqueous further extracted with EtOAc. The combined organic phases were washed with brine, dried (MgSO₄) and the solvent removed in vacuo. The crude mixture was purified by flash chromatography with 10% EtOAc/Hexane as eluent to afford the title compound (0.526 g, 84%): RT=4.09 min; m/z (ES⁺)=293.17 [(M-15)+H]⁺

Intermediate 7: 4-(4-Methanesulfonylphenyl)piperidine-1-carboxylic acid tert-butyl ester

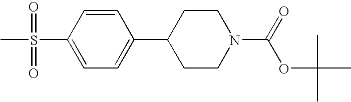

To a solution of 4-(4-methylsulfanylphenyl)piperidine-1 carboxylic acid tert-butyl ester (0.25 g, 0.813 mmol) in DCM (10° mL) was added mCPBA (0.383 g, 1.71 mmol) at rt. The solution was allowed to stir for 2.5 h. The reaction mixture was diluted with DCM (20 mL), washed with saturated Na₂CO₃ solution, dried (MgSO₄) and the solvent removed in vacuo to yield the title compound (0.284 g, 100%): RT=3.39 min; m/z (ES⁺)=339.5 [M+H]⁺

### Example 55

### 4-{2-[4-(4-Methanesulfonylphenyl)piperidin-1-yl]ethyl}piperidine-1-carboxylic acid tert-butyl ester

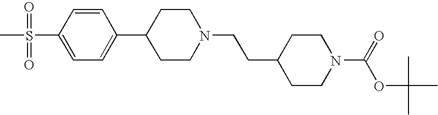

A solution of 4-(4-methanesulfonylphenyl)piperidine-1-carboxylic acid tert-butyl ester (0.276 g, 0.813 mmol) in DCM (15 mL) was treated with TFA (1.5 mL) and the mixture stirred at rt for 0.5 h. DCM (30 mL) was added and the organic layer washed with saturated Na₂CO₃ solution, brine, dried (MgSO₄) and the solvent removed in vacuo to yield 4-(4-methanesulfonylphenyl)piperidine (0.19 g, 97%). To a solution of the solid (0.189 g, 0.79 mmol) in MeOH (5 mL) was added N-boc-piperidinyl-4-acetaldehyde (0.215 g, 0.95 mmol) and the mixture allowed to stir at rt for 20 h. The reaction was cooled to 0° C. and treated with sodium borohydride (0.045 g, 1.18 mmol). The reaction was stirred for 1 h and the solvent removed in vacuo. DCM (25 mL) and water (10 mL) were added and the two layers separated. The aqueous phase was further extracted with DCM and the combined organic phases washed with brine, dried (MgSO₄) and the solvent removed in vacuo. The crude mixture was purified by flash chromatography with 1% NEt₃, 2% MeOH/EtOAc as eluent to afford the title compound (0.252 g, 79%): RT=2.80 min; m/z (ES⁺)=451.4 [M+H]⁺

### Example 56

### 4-{2-[4-(4-Methylsulfanylphenyl)piperidin-1-yl]ethyl}piperidine-1-carboxylic acid tert-butyl ester

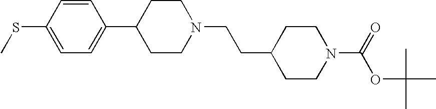

To a solution of 4-(4-methylsulfanylphenyl)piperidine hydrochloride (0.244 g, 1.00 mmol) in MeOH (10 mL) was added NEt₃ (0.14 mL, 1 mmol) followed by N-boc-piperidinyl-4-acetaldehyde (0.273 g, 1.2 mmol). The mixture was allowed to stir at rt for 20 h. The reaction was cooled to 0° C. and treated with sodium borohydride (0.057 g, 1.5 mmol). The reaction was stirred for 1 h and the solvent removed in vacuo. DCM (30 mL) and water (20 mL) was added and the two layers separated. The aqueous phase was further extracted with DCM and the combined organic phases washed with brine, dried (MgSO₄) and the solvent removed in vacuo. The crude mixture was purified by flash chromatography with EtOAc as eluent to afford the title compound (0.132 g, 32%): RT=2.86 min; m/z (ES⁺)=418.6 [M+H]⁺

Intermediate 8: (1S,4S)-2-(4-Methanesulfonylphenyl)-2,5-diazabicyclo[2.2.1]heptane

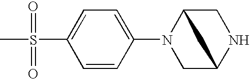

A mixture of 1-fluoro-4-methanesulfonylbenzene (0.697 g, 4.0 mmol), (1S,4S)-2,5-diazabicyclo[2.2.1]heptane (2.0 g, 20.0 mmol) and K₂CO₃ (5.33 g, 40.0 mmol) in DMF (30 mL) was heated at 150° C. for 4 h. The solvent was removed in vacuo and the resulting solid dissolved in DCM (30 mL). The organic phased was washed with water, brine, dried (MgSO₄) and the solvent removed in vacuo. The crude mixture was purified by flash chromatography with 50% MeOH/EtOAc as eluent to afford the title compound (0.374 g, 37%): RT=1.81 min; m/z (ES⁺)=253.1 [M+H]⁺

Intermediate 9: (S)-1-(4-Methanesulfonylphenyl)-3-methylpiperazine

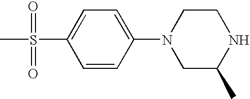

A mixture of 1-fluoro-4-methanesulfonylbenzene (0.74 g, 425 mmol) and (S)-2-methyl piperazine (2.13 g, 21.3 mmol) was heated at 150° C. for 2 h. The reaction was cooled and DCM and water was added. The two layers were separated and the organic phase washed with water, brine, dried (MgSO₄) and the solvent removed in vacuo to afford the title compound (0.916 g, 85%): RT=1.64 min; m/z (ES⁺)=255.1 [M+H]⁺

The compounds shown in Table 7 below were synthesised by analogous methods from the appropriate amine:

TABLE 7

| | | | RT | m/z |
|---|---|---|---|---|
| Int | Structure | Name | (min) | (ES+) |
| 10 | 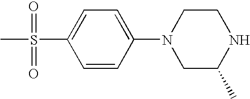 | (R)-1-(4-Methanesulfonyl phenyl)-3- methylpiperazine | 1.64 | 255.1 |
| 11 | 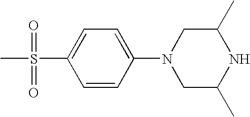 | 1-(4-Methanesulfonylphenyl)-3,5- dimethylpiperazine | 1.71 | 269.15 |
| 12 | 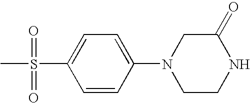 | 4-(4-Methanesulfonylphenyl) piperazin-2-one | 2.16 | 255.0 |
| 13 | 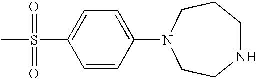 | 1-(4-Methanesulfonylphenyl) [1,4]diazepane | 1.71 | 255.0 |

### Example 57

### 4-{2-[(S)-4-(4-Methanesulfonylphenyl)-2-methylpiperazin-1-yl]ethyl}piperidine-1-carboxylic acid tert-butyl ester

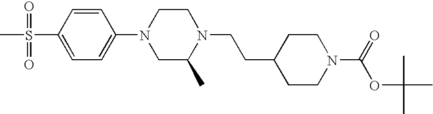

A solution of (S)-1-(4-methanesulfonylphenyl)-3-methylpiperazine (0.387 g, 1.52 mmol) and N-Boc-piperidinyl-4-acetaldehyde (0.692 g. 3.05 mmol) in MeOH (10 mL) was allowed to stir at room temperature for 20 h. The mixture was cooled to 0° C. and treated with sodium borohydride (0.191 g, 5.03 mmol). The reaction was stirred for an additional 1 h and the solvent removed in vacuo. EtOAc (25 mL) and water (10 mL) was added and the two layers separated. The aqueous phase was further extracted with DCM and the combined organic phases washed with brine, dried (MgSO₄) and the solvent removed in vacuo. The crude mixture was purified by flash chromatography with 5% NEt₃/EtOAc as eluent to afford the title compound (0.047 g, 7%): RT=2.59 min; m/z (ES⁺)=466.4 [M+H]⁺

The compounds shown in Table 8 below were synthesised by analogous methods from the appropriate aldehyde and amine:

TABLE 8

| | | | RT | m/z |
|---|---|---|---|---|
| Eg | Structure | Name | (min) | (ES+) |
| 58 | 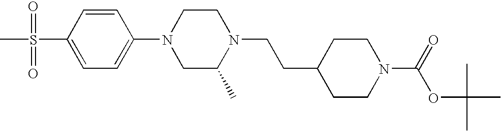 | 4-{2-[(R)-4-(4- Methanesulfonyl-phenyl)-2- methyl-piperazin-1-yl]-ethyl}- piperidine-1-carboxylicacid tert-butyl ester | 2.75 | 466.4 |
| 59 | 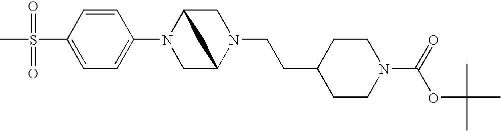 | 4-{2-[(1S,4S)-5-(4- Methanesulfonyl-phenyl)-2,5- diaza-bicyclo[2.2.1]hept-2- yl]-ethyl}-piperidine-1- carboxylic acid tert-butyl ester | 2.62 | 464.31 |
| 60 | 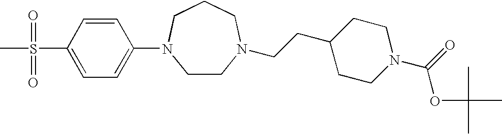 | 4-{2-[4-(4-Methanesulfonyl- phenyl)-[1,4]diazepan-1-yl]- ethyl}-piperidine-1-carboxylic acid tert-butyl ester | 2.57 | 466.4 |
| 61 | 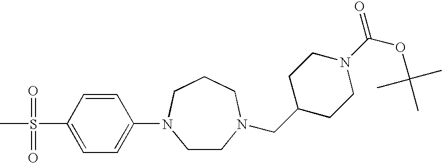 | 4-[4-(4-Methanesulfonyl- phenyl)-[1,4]diazepan-1- ylmethyl]-piperidine-1- carboxylic acid tert-butyl ester | 2.53 | 452.4 |

### Example 62

### 4-{2-[4-(4-Methanesulfonylphenyl)-2,6-dimethylpiperazin-1-yl]ethyl}piperidine-1-carboxylic acid tert-butyl ester

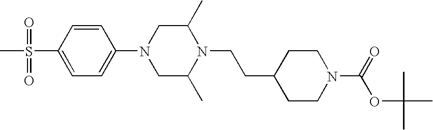

To a solution of 1-(4-methanesulfonylphenyl)-3,5-dimethylpiperazine (0.067 g, 0.25 mmol) in MeCN (2 mL) was added 4-(2-methanesulfonyloxyethyl)piperidine-1-carboxylic acid tert-butyl ester (0.079 g, 0.25 mmol) and K₂CO₃ (0.038 g, 0.275 mmol). The mixture was heated to reflux and allowed to stir for 20 h. EtOAc (10 mL) was added and the organic layer washed with water, brine, dried (MgSO₄) and the solvent removed in vacuo. The crude mixture was purified by flash chromatography with 100% MeOH/EtOAc as eluent to afford the title compound (0.006 g, 5%): RT=2.76 min; m/z (ES⁺)=480.4 [M+H]⁺

### Example 63

### 2-[4-(4-methanesulfonylphenyl)-2-oxopiperazin-1-yl]ethyl}piperidine-1-carboxylic acid tert-butyl ester

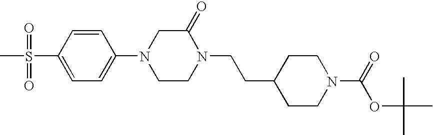

To a solution of 4-(4-methanesulfonylphenyl)piperazin-2-one (0.037 g, 0.144 mmol) in anhydrous DMF (1 mL) was added sodium hydride (0.0065 g of a 60% dispersion in mineral oil, 0.164 mmol) at rt. The solution was allowed to stir for 30 min then treated with 4-(2-methanesulfonyloxyethyl)piperidine-1-carboxylic acid tert-butyl ester (0.044 g, 0.144 mmol) and allowed to stir for a further 20 h. The solvent was removed in vacuo and the residue dissolved in EtOAc (10 mL), washed with water, brine, dried (MgSO₄) and the solvent removed in vacuo. The crude mixture was purified by flash chromatography with 50% EtOAc/Hexane as eluent to afford the title compound (0.007 g, 10%): RT=3.34 min; m/z (ES⁺)=466.2 [M+H]+

Intermediate 14: 2-(2-Hydroxyethylamino)-N-(4-methylsulfanylphenyl)acetamide

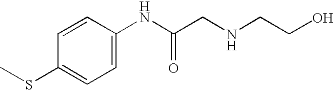

To a solution of 4-methylsulfanylphenylamine (2.5 g, 17.96 mmol) in iso-propyl acetate (37 mL) was added a solution of KHCO₃ (3.147 g, 31.4 mmol) in water (15 mL). The reaction was cooled to 0° C. and treated with 2-chloroacetylchloride (1.76 mL, 22.1 mmol) dropwise. The reaction was allowed to warm to rt over 1 h and the two layers separated. The organic phase was washed with water, brine, dried (MgSO₄) and the resulting solution treated with ethanolamine (4.34 mL, 71.9 mmol). The reaction was heated at 60° C., after which the solvent was removed and the residue purified by flash chromatography with 5% NEt₃/10% MeOH/EtOAc, then recrystallised from EtOAc the afford the title compound (1.234 g, 29%): RT=1.99 min; m/z (ES⁺)=241.0 [M+H]⁺

### Intermediate 15: 1-(4-Methylsulfanylphenyl)piperazin-2-one

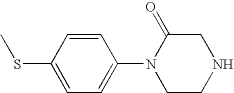

To a solution of 2-(2-hydroxyethylamino)-N-(4-methylsulfanylphenyl)acetamide (0.6 g, 2.5 mmol) in EtOAc (4 mL) was added P(n-Bu)₃ (0.812 mL, 3.25 mmol) at 0° C. After 5 min a solution of DBAD (0.748 g, 3.25 mmol) in EtOAc (20 mL) was added dropwise. The solution was allowed to warm to rt then stirred at 40° C. for 3 days. The solvent was removed in vacuo and the crude mixture purified by flash chromatography with EtOAc as eluent to afford the title compound (0.235 g, 42%): RT=0.93 min; m/z (ES⁺)=223.04 [M+H]⁺

### Example 64

### 4-{2-[4-(4-Methylsulfanylphenyl)-3-oxopiperazin-1-yl]ethyl}piperidine-1-carboxylic acid tert-butyl ester

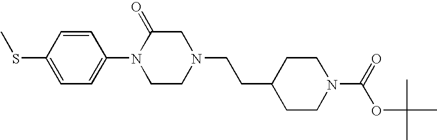

To a solution of 1-(4-methylsulfanylphenyl)piperazin-2-one (12.0 g, 39.0 mmol) in MeCN (200 mL) was added K₂CO₃ (0.157 g, 1.14 mmol), tetrabutylammonium iodide (0.926 g, 2.51 mmol) and 4-(2-methanesulfonyloxyethyl)piperidine-1-carboxylic acid tert-butyl ester (15.46 g, 50.2 mmol) and the mixture heated at reflux for 3 days. The solvent was removed in vacuo and the residue dissolved in EtOAc (100 mL), washed with water, brine, dried (MgSO₄) and the solvent removed in vacuo. The crude mixture purified by flash chromatography with EtOAc as eluent to afford the title compound (4.578 g, 42%): RT=2.70 min; m/z (ES⁺)=434.19 [M+H]⁺

### Example 65

### 4-{2-[4-(4-Methanesulfonylphenyl)-3-oxopiperazin-1-yl]ethyl}piperidine-1-carboxylic acid tert-butyl ester

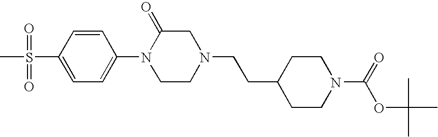

To a solution of 4-{2-[4-(4-methylsulfanylphenyl)-3-oxo-piperazin-1-yl]ethyl}-piperidine-1-carboxylic acid tert-butyl ester (4.578 g, 10.5 mmol) in toluene (100 mL) was added NaMoO₄ (0.508 g, 2.1 mmol) followed by N(n-Bu)₃ (0.251 mL, 1.05 mmol). Acetic acid (0.67 mL) was added followed by 30% H₂O₂/H₂O (0.52 mL). Further portions of acetic acid and H₂O₂ were added at 5 min intervals until no red precipitate was observed. The reaction was treated with saturated Na₂SO₃ solution and the aqueous extracted with EtOAc. The combined organic layers were dried (MgSO₄) and the solvent removed in vacuo. The crude mixture was purified by flash chromatography with 5% MeOH EtOAc as eluent to afford the title compound (0.837 g, 17%): RT=2.51 min; m/z (ES⁺)=466.15 [M+H]⁺

### Example 66

### 4-{2-[4-(4-Methanesulfinylphenyl)-3-oxopiperazin-1-yl]ethyl}piperidine-1-carboxylic acid tert-butyl ester

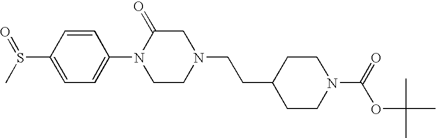

Title compound isolated from previous reaction (1.351 g, 29%): RT=2.56 min; m/z (ES⁺)=450.15 [M+H]⁺

### Example 67

### 4-{2-[4-(4-Methanesulfonylphenyl)piperazin-1-yl]acetyl}piperidine-1-carboxylic acid tert-butyl ester

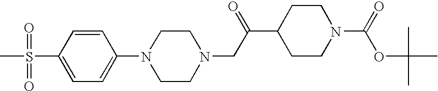

To a solution of 1-(4-methanesulfonylphenyl)piperazine (0.055 g, 0.23 mmol) in MeCN (2 mL) was added 4-(2-bromoacetyl)piperidine-1-carboxylic acid tert-butyl ester (0.07 g, 0.23 mmol) and K₂CO₃ (0.035 g, 0.25 mmol). The mixture was heated at reflux for 4 h then allowed to cool. EtOAc (20 mL) was added and the organic phase washed with water, brine, dried (MgSO₄) and the solvent removed in vacuo. The crude mixture was purified by flash chromatography with EtOAc as eluent to afford the title compound (0.07 g, 65%): RT=2.45 min; m/z (ES⁺)=466.4 [M+H]⁺

### Example 68

### 4-{2-[4-(3-Fluoro-4-methylsulfanylphenyl)piperazin-1-yl]acetyl}piperidine-1-carboxylic acid tert-butyl ester

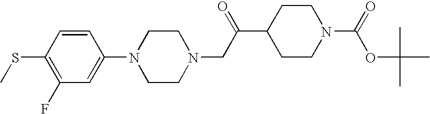

Prepared using the above method: RT=2.83 min; m/z (ES⁺)=452.3 [M+H]⁺

### Example 69

### 4-{2-[4-(3-Fluoro-4-methanesulfonylphenyl)piperazin-1-yl]acetyl}piperidine-1-carboxylic acid tert-butyl ester

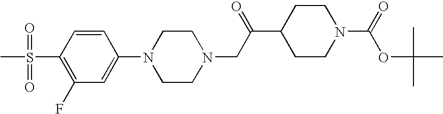

To a solution of 4-{2-[4-(3-fluoro-4-methylsulfanylphenyl)piperazin-1-yl]acetyl}piperidine-1-carboxylic acid tert-butyl ester (0.13 g, 0.29 mmol) in toluene (2 mL) was added NaMoO₄ (0.007 g, 0.03 mmol) followed by N(n-Bu)₃ (0.007 mL, 0.03 mmol). Acetic acid (3×0.018 mL) was added, followed by 27% H₂O₂/H₂O (0.015 mL). To the red oily residue, was added acetic acid (7×0.018 mL) followed by 27% H₂O₂/H₂O (4×0.015 mL). The mixture was stirred at rt for 30 min and then quenched with saturated Na₂SO₃ solution. The aqueous was taken to pH8 with 1M NaOH and extracted with EtOAc. The combined organic layers were dried (MgSO₄) and the solvent removed in vacuo. The crude mixture was purified by flash chromatography with 10% MeOH/EtOAc as eluent to afford the title compound (0.08 g, 60%): RT=2.54 min; m/z (ES⁺)=484.2 [M+H]⁺

### Example 70

### 4-{2-[4-(3-Fluoro-4-methanesulfinylphenyl)piperazin-1-yl]acetyl}piperidine-1-carboxylic acid tert-butyl ester

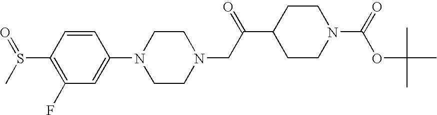

Isolated from previous reaction: RT=2.42 min; m/z (ES⁺)=468.2 [M+H]⁺

### Example 71

### 4-{1,1-Difluoro-2-[4-(4-methanesulfonylphenyl)piperazin-1-yl]ethyl}piperidine-1-carboxylic acid tert-butyl ester

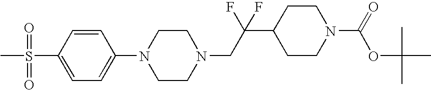

To a solution of 4-{2-[4-(4-methanesulfonylphenyl)piperazin-1-yl]acetyl}piperidine-1-carboxylic acid tert-butyl ester (0.04 g, 0.09 mmol) in DCM (0.6 mL) was added DAST (0.4 mL, 3.1 mmol) and the reaction stirred at rt for 2 h. The reaction was cooled to 0° C. and quenched with water. The two layers were separated and the organic layer washed with brine, dried (MgSO₄) and the solvent removed in vacuo. The crude mixture was purified by flash chromatography with 50% EtOAc/Hexane as eluent to afford the title compound (0.017 g, 40%): RT=3.10 min; m/z (ES⁺)=488.3 [M+H]⁺

### Example 72

### 4-{1,1-Difluoro-2-[4-(3-fluoro-4-methanesulfonylphenyl)piperazin-1-yl]ethyl}piperidine-1-carboxylic acid tert-butyl ester

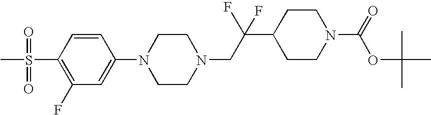

Prepared using the above method: RT=3.36 min; m/z (ES⁺)=506.2 [M+H]⁺

### Example 73

### 4-{1-Hydroxy-2-[4-(4-methanesulfonylphenyl)piperazin-1-yl]ethyl}piperidine-1-carboxylic acid tert-butyl ester

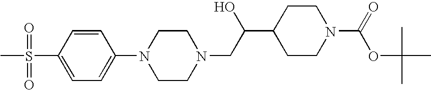

To a solution of 4-{2-[4-(4-methanesulfonylphenyl)piperazin-1-yl]acetyl}piperidine-1-carboxylic acid tert-butyl ester (0.1 g, 0.215 mmol) in THF (5 mL) was added sodium borohydride (0.016 g, 0.42 mmol) and the mixture was allowed to stir at rt for 20 h. The solvent was removed in vacuo and the crude mixture purified by flash chromatography with EtOAc as eluent to afford the title compound (0.07 g, 70%): RT=2.32 min; m/z (ES⁺)=468.2 [M+H]⁺

### Example 74

### 4-{(1-Chloro-2-[4-(4-methanesulfonylphenyl)piperazin-1-yl]ethyl}piperidine-1-carboxylic acid tert-butyl ester

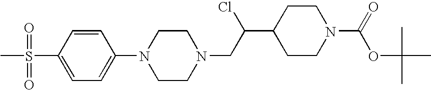

To a solution of DAST (0.06 mL, 0.43 mmol) in DCM (0.5 mL) at −55° C. was added a solution of 4-{1-hydroxy-2-[4-(4-methanesulfonylphenyl)piperazin-1-yl]ethyl}piperidine-1-carboxylic acid tert-butyl ester (0.1 g, 0.21 mmol) in DCM (0.5 mL). The reaction was allowed to warm to 5° C. over 3 h. The reaction was quenched with water. The two layers were separated and the organic layer was washed with brine, dried (MgSO₄) and the solvent removed in vacuo. The crude mixture was purified by flash chromatography with 50% EtOAc/Hexane as eluent to afford the title compound (0.025 g, 24%): RT=2.60 min; m/z (ES⁺)=486.2 [M+H]⁺

### Example 75

### 4-{1-Fluoro-2-[4-(4-methanesulfonylphenyl)piperazin-1-yl]ethyl}piperidine-1-carboxylic acid tert-butyl ester

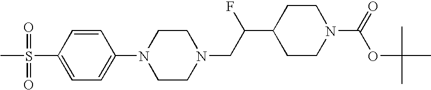

To a solution of 4-{1-hydroxy-2-[4-(4-methanesulfonylphenyl)piperazin-1-yl]ethyl}piperidine-1-carboxylic acid tert-butyl ester (0.04 g, 0.08 mmol) in DCM (1 mL) was added DAST (0.393 mL, 2.98 mmol) and the reaction stirred at rt for 0.5 h. The reaction was cooled to 0° C. and quenched with water. The two layers were separated and the organic layer washed with brine, dried (MgSO₄) and the solvent removed in vacuo. The crude mixture was purified by flash chromatography with 50% EtOAc/DCM as eluent to afford the title compound (0.003 g, 7%): RT=2.39 min; m/z (ES⁺)=470.2 [M+H]⁺

### Example 76

### 4-{2-Fluoro-1-[4-(4-methanesulfonylphenyl)piperazin-1-yl]ethyl}piperidine-1-carboxylic acid tert-butyl ester

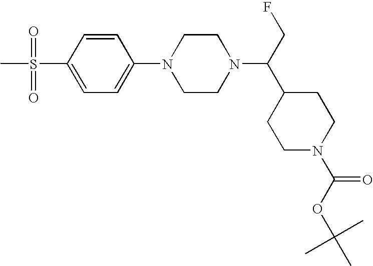

To a solution of 4-{1-hydroxy-2-[4-(4-methanesulfonylphenyl)piperazin-1-yl]ethyl}piperidine-1-carboxylic acid tert-butyl ester (0.05 g, 1.1 mmol) in DCM (5 mL) was added DAST (0.393 mL, 2.98 mmol) and the reaction stirred at rt for 0.7 h. The reaction was cooled to 0° C. and quenched with water. The two layers were separated and the organic layer washed with brine, dried (MgSO₄) and the solvent removed in vacuo. The crude mixture was purified by flash chromatography with 50% EtOAc/DCM as eluent to afford the title compound (0.003 g, 6%): RT=2.70 min; m/z (ES⁺)=470.2 [M+H]⁺

Intermediate 16: 4-(2-Hydroxyethylidene)piperidine-1-carboxylic acid tert-butyl ester

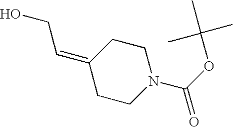

To a solution of 4-ethoxycarbonylmethylenepiperidine-1-carboxylic acid tert-butyl ester (3.5 g, 13.01 mmol) in toluene (30 mL) at −78° C. was added DIBAL (33 mL of a 1M solution in toluene, 33.0 mmol) dropwise. The mixture was stirred at −78° C. for 1 h then treated with MeOH (0.5 mL) and allowed to warm to rt. Water was added and the precipitate removed by filtration. The filtrate was concentrated in vacuo and the crude mixture purified by flash chromatography with 33% EtOAc/Hexane as eluent to afford the title compound as a yellow oil (2.2 g, 75%): δ_{H} (CDCl₃) 1.30 (9H, s), 2.02 (2H, m), 2.10 (2H, m), 3.22 (4H, m), 4.01 (2H, m), 5.35 (1H, t).

### Example 77

### 4{-2-[4-(4-Methanesulfonylphenyl)piperazin-1-yl]-ethylidene}piperidine-1-carboxylic acid tert-butyl ester

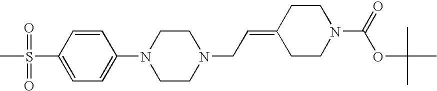

To a solution of 4-(2-hydroxyethylidene)piperidine-1-carboxylic acid tert-butyl ester (2.2 g, 9.7 mmol) in DCM (25 mL) was added Et₃N (2.02 mL, 14.5 mmol) and the reaction cooled to 0° C. To this cooled mixture was added methanesulfonylchloride (0.98 mL, 12.6 mmol) dropwise. The reaction was stirred at 0° C. for 20 min then treated with saturated NaHCO3 solution. The two layers were separated and the organic layer washed with water, brine, dried (MgSO₄) and the solvent removed in vacuo. The crude mixture was purified by flash chromatography with 10% EtOAc/Hexane as eluent to afford 4-(2-chloroethylidene) piperidine-1-carboxylic acid tert-butyl ester and 4-vinyl-3,6-dihydro-2H-pyridine-1-carboxylic acid tert-butyl ester in a 1:1 ratio (0.950 g). The mixture was dissolved in DMF (5 mL) and treated with TBAI (0.068 g, 0.18 mmol). This suspension was thus added to a preformed mixture of 1-(4-methanesulfonylphenyl)piperazine (0.487 g, 2.03 mmol) and sodium hydride (0.11 g of a 60% dispersion in mineral oil, 2.77 mmol) in DMF (5 mL) at rt. The mixture was allowed to stir for 2 h then treated with water. The aqueous was extracted with EtOAc and the combined organic layers washed with water, brine, dried (MgSO₄) and the solvent removed in vacuo. The crude mixture was purified by HPLC to afford the title compound (0.27 g, 6%): RT=2.41 min; m/z (ES⁺)=450.2 [M+H]⁺

Intermediate 17: 4-[2-(4-Oxopiperidin-1-yl)ethyl]piperidine-1-carboxylic acid tert-butyl ester

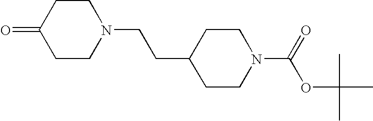

To a solution of piperidin-4-one (0.091 g, 0.59 mmol) in MeCN (3.5 mL) was added K₂CO₃ (0.179 g, 1.3 mmol) and 4-(2-methanesulfonyloxyethyl)piperidine-1-carboxylic acid tert-butyl ester (G. A. Cain et. al., U.S. Pat. No. 5,252,586) (0.2 g, 1.3 mmol). The solution was allowed to stir at rt for 20 h, then at reflux for a further 6 h. Water was added followed by EtOAc. The two layers were separated and the aqueous further extracted with EtOAc. The combined organic layers were dried (MgSO₄) and the solvent removed in vacuo. The crude mixture was purified by flash chromatography with 50% EtOAc/Hexane as eluent to afford the title compound (0.077 g, 42%): RT=2.07 min; m/z (ES⁺)=311.3 [M+H]⁺

### Example 78

### 4-{2-[4-Hydroxy-4-(4-methylsulfanylphenyl)piperidin-1-yl]ethyl}piperidine-1-carboxylic acid tert-butyl ester

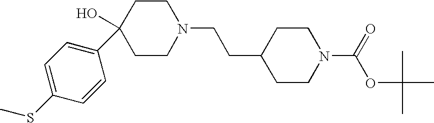

To a solution of 4-[2-(4-oxopiperidin-1-yl)ethyl]piperidine-1-carboxylic acid tert-butyl ester (0.077 g, 0.248 mmol) in anhydrous THF (1.2 mL) at 0° C. was added thiomethylbenzene magnesium bromide (0.5 mL of a 0.5 Mol solution in THF, 0.25 mmol). The solution was stirred at 0° C. for 30 mins then treated with saturated NH₄Cl solution followed by EtOAc. The two layers were separated and the aqueous layer further extracted with EtOAc. The combined organic layers were dried (MgSO₄) and the solvent removed in vacuo. The crude mixture was purified by flash chromatography with 50% EtOAc/Hexane as eluent to afford the title compound (0.074 g, 69%): RT=2.76 min; m/z (ES⁺)=435.35 [M+H]⁺

### Example 79

### 4-{2-[4-Hydroxy-4-(4-methanesulfonylphenyl)piperidin-1-yl]ethyl}piperidine-1-carboxylic acid tert-butyl ester

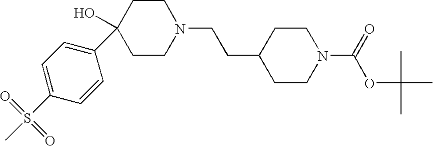

To a solution of 4-{2-[4-hydroxy-4-(4-methylsulfanylphenyl)piperidin-1-yl]ethyl}piperidine-1-carboxylic acid tert-butyl ester (0.071 g, 0.164 mmol) in toluene (1 mL) was added NaMoO₄ (0.0039 g, 0.016 mmol) followed by N(n-Bu)₃ (0.004 mL, 0.016 mmol). Acetic acid (0.010 mL) was added followed by H₂O₂ (0.010 mL). Further portions of acetic acid and H₂O₂ were added at 5 min intervals (4×0.010 mL) and the mixture heated at 60° C. for 15 min. The reaction was treated with saturated Na₂SO₃ solution and the aqueous extracted with EtOAc. The combined organic layers were dried (MgSO₄) and the solvent removed in vacuo. The crude mixture was purified by flash chromatography with 2% NH₃, 5% MeOH/DCM as eluent to afford the title compound (0.035 g, 46%): RT=2.39 min; m/z (ES⁺)=467.35 [M+H]⁺

### Example 80

### 4-{2-[4-(4-Methanesulfonylphenyl)piperazin-1-yl]-2-oxoethyl}piperidine-1-carboxylic acid tert-butyl ester

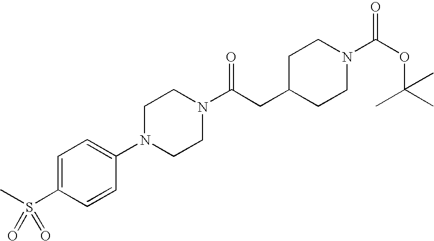

To a solution of 1-(4-methanesulfonylphenyl)piperazine (0.22 g, 0.91 mmol), 4-carboxy methylpiperidine-1-carboxylic acid tert-butyl ester (0.20 g, 0.80 mmol), HOBT.H₂O (0.14 g, 0.91 mmol) and DIPEA (0.47 mL, 2.72 mmol) in DMF (5 mL) was added EDCI (0.19 g, 0.99 mmol) and the mixture was stirred for 18 h. The solvent was removed under vacuum and the resulting residue was partitioned between EtOAc and saturated NaHCO₃ solution. The aqueous phase was re-extracted with EtOAc, the organic extracts were combined, washed with brine, dried (MgSO₄) and adsorbed onto SiO₂. The adsorbed sample was purified by flash chromatography eluting with 50:50 EtOAc:hexane to afford the title compound: RT=3.26 min; m/z (ES⁺)=466.33 [M+H]⁺.

### Example 81

### 4-{2-[4-(4-Methanesulfonylphenyl)piperazin-1-yl]-2-oxoethyl}piperidine-1-carboxylic acid tert-butyl ester

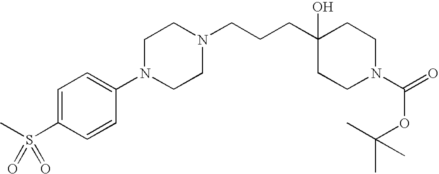

To a solution of 4-hydroxy-4-(3-hydroxypropyl)piperidine-1-carboxylic acid tert-butyl ester (1.00 g, 3.86 mmol) in DCM (60 mL) was added Dess-Martin periodinane (1.80 g, 4.24 mmol) and the mixture was stirred for 1 h at rt, a further batch of Dess-Martin periodinane (0.20 g, 0.47 mmol). The reaction mixture was quenched with 2 M NaOH and extracted with Et₂O, the aqueous phase was re-extracted with Et₂O and the organic extracts were combined then washed with water, 2 M NaOH solution and brine, dried (MgSO₄) and the solvent was removed under vacuum to give 2-hydroxy-1-oxa-8-azaspiro[4.5]decane-8-carboxylic acid tert-butyl ester.

A solution of 1-(4-methanesulfonylphenyl)piperazine (0.12 g, 0.50 mmol) and 2-hydroxy-1-oxa-8-azaspiro[4.5]decane-8-carboxylic acid tert-butyl ester (0.14 g, 0.56 mmol) in anhydrous MeOH (2 mL) was heated at 75° C. for 1 h, then NaBH₄ (25 mg, 0.65 mmol) was added and the reaction was stirred for 2 h. The solvent was removed under vacuum and the resulting residue was partitioned between water and DCM. The aqueous phase was re-extracted with DCM, the organic extracts were combined and purified by flash chromatography eluting with 3:97 MeOH:DCM to afford the title compound. RT=2.37 min; m/z (ES⁺)=482.45 [M+H]⁺.

Intermediate 18: 4-(6-Chloropyridin-3-yl)piperazine-1-carboxylic acid tert-butyl ester

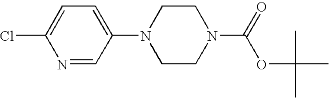

A mixture of 2-chloro-5-bromopyridine (1.0 g, 5.2 mmol), 1-boc-piperazine (0.967 g, 5.2 mmol), sodium tert-butoxide (0.749 g, 7.8 mmol), 9,9-dimethyl-4,5-bis(diphenylphosphino)xanthene (0.179 g, 0.31 mmol) in toluene (30 mL) was treated with Pd₂(dba)₃ (0.095 g, 0.1 mmol) at rt. The mixture was refluxed for 4 h. The reaction was cooled and filtered through celite. The organic layer was diluted with EtOAc (100 mL) then washed with saturated Na₂CO₃ solution, brine, dried (MgSO₄) and the solvent removed in vacuo. The crude mixture was purified by flash chromatography with 20% EtOAc/Hexane as eluent to afford the title compound (0.82 g, 53%): RT=3.40 min; m/z (ES⁺)=298.2 [M+H]⁺

### Example 82

### 4-{2-[4-(6-Chloropyridin-3-yl)piperazin-1-yl]ethyl}piperidine-1-carboxylic acid tert-butyl ester

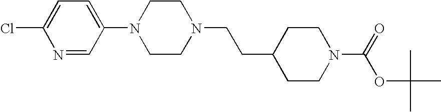

A solution of 4-(6-chloropyridin-3-yl)piperazine-1-carboxylic acid tert-butyl ester (0.15 g, 0.5 mmol) in DCM (5 mL) was treated with TFA (1 mL) and the mixture stirred at rt for 4 h. DCM (20 mL) was added and the organic layer washed with 2M NaOH solution, brine, dried (MgSO₄) and the solvent removed in vacuo to yield 1-(6-chloro pyridin-3-yl)piperazine as a yellow solid (0.067 g, 68%). The solid was dissolved in DCM (8 mL) and treated with N-boc-piperidinyl-4-acetaldehyde (0.077 g, 0.34 mmol) and 4A molecular sieves (0.1 g) at rt. The solution was allowed to stir for 1 h then treated with NaHB(OAc)₃ (0.094 g, 0.44 mmol). The resulting solution was stirred at rt for 24 h. DCM was added and the organic layer washed with saturated Na₂CO₃ solution, brine, dried (MgSO₄) and the solvent removed in vacuo. The crude material was purified by flash chromatography with EtOAc as eluent to afford the title compound (0.098 g, 70%): RT=2.56 min; m/z (ES⁺)=409.3 [M+H]⁺

Intermediate 19: 4-(6-Methylsulfanylpyridin-3-yl)piperazine-1-carboxylic acid tert-butyl ester

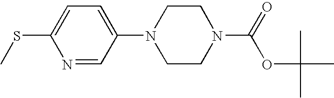

A mixture of 5-bromo-2-methylsulfanylpyridine (1.06 g, 5.2 mmol), 1-boc-piperazine (0.967 g, 5.2 mmol), sodium tert-butoxide (0.749 g, 7.8 mmol), 9,9-dimethyl-4,5-bis(diphenylphosphino)xanthene (0.179 g, 0.31 mmol) in toluene (30 mL) was treated with Pd₂(dba)₃ (0.095 g, 0.1 mmol) at rt. The mixture was refluxed for 3 h. The reaction was cooled and filtered through celite. The organic layer was diluted with EtOAc (100 mL) then washed with saturated Na₂CO₃ solution, brine, dried (MgSO₄) and the solvent removed in vacuo. The crude mixture was purified by flash chromatography with 20% EtOAc/Hexane as eluent to afford the title compound (1.0 g, 61%): RT=3.22 min; m/z (ES⁺)=310.2 [M+H]⁺

Intermediate 20: 4-(6-Methanesulfonylpyridin-3-yl)piperazine-1-carboxylic acid tert-butyl ester

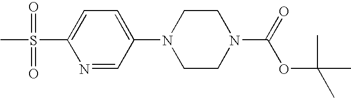

To a solution of 4-(6-methylsulfanylpyridin-3-yl)piperazine-1-carboxylic acid tert-butyl ester (0.5 g, 1.62 mmol) in DCM (20 mL) at 0° C. was added mCPBA (0.56 g, 3.24 mmol) portionwise. The mixture was allowed to warm to rt and stir for 3 h. DCM (30 mL) was added and the organics washed with saturated Na₂CO₃ solution, dried (MgSO₄) and the solvent removed in vacuo. The crude mixture was purified by flash chromatography with 80% EtOAc/Hexane as eluent to afford the title compound (0.14 g, 25%): RT=3.07 min; m/z (ES⁺)=342.2 [M+H]⁺

### Example 83

### 4-{2-[4-(6-Methanesulfonylpyridin-3-yl)piperazin-1-yl]ethyl}piperidine-1-carboxylic acid tert-butyl ester

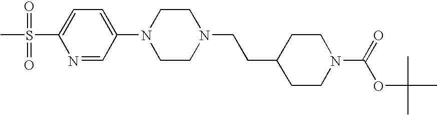

A solution of 4-(6-methanesulfonylpyridin-3-yl)piperazine-1-carboxylic acid tert-butyl ester (0.14 g, 0.4 mmol) in DCM (10 mL) was treated with TFA (1 mL) and the mixture stirred at rt for 3 h. DCM (30 mL) was added and the organic layer washed with 1M NaOH solution, brine, dried (MgSO₄) and the solvent removed in vacuo to yield 1-(6-methanesulfonylpyridin-3-yl)piperazine as a yellow solid (0.095 g, 100%). The solid was dissolved in DCM (8 mL) and treated with N-boc-piperidinyl-4-acetaldehyde (0.088 g, 0.39 mmol) and 4A molecular sieves (0.1 g) at room temperature. The solution was allowed to stir for 3 h then treated with NaHB(OAc)₃ (0.106 g, 0.5 mmol). The resulting solution was stirred at rt for 20 h. DCM (20 mL) was added and the organic layer washed with saturated Na₂CO₃ solution, brine, dried (MgSO₄) and the solvent removed in vacuo. The crude material was purified by flash chromatography with EtOAc as eluent to afford the title compound (0.101 g, 58%): RT=2.61 min; m/z (ES⁺)=453.4 [M+H]⁺

Intermediate 21: 2-Bromo-5-methanesulfonylpyridine

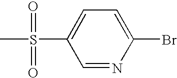

To a solution of 2-bromo-5-methylsulfanylpyridine (1.53 g, 7.6 mmol) in DCM (20 mL) at 0° C. was added mCPBA (3.95 g, 15.95 mmol) portionwise. The mixture was allowed to warm to rt and stir for 2 h. DCM (30 ml) was added and the organics washed with saturated Na₂SO₃ solution, saturated Na₂CO₃ solution, dried (MgSO₄) and the solvent removed in vacuo. The crude product was triturated with Et₂O, filtered and dried in vacuo to afford the title compound (1.25 g, 71%): δ_{H} (CDCl₃) 3.15 (3H, s), 7.75 (1H, d), 8.08 (1H, dd), 8.95 (1H, d).

Intermediate 22: 4-(5-Methanesulfonylpyridin-2-yl)piperazine-1-carboxylic acid tert-butyl ester

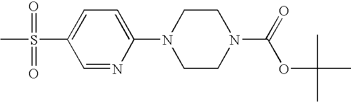

A solution of 2-bromo-5-methanesulfonylpyridine (2.1 g, 8.9 mmol) and 1-boc-piperazine (3.31 g, 17.8 mmol) in trifluoroethanol (25 mL) was heated at reflux for 24 h. The solvent was removed in vacuo and EtOAc (100 mL) added. The solution was washed with water, brine, dried (MgSO₄) and the solvent removed in vacuo. The crude mixture was purified by flash chromatography with 40% EtOAc/Hexane as eluent to afford the title compound (1.86 g, 62%): RT=3.11 min; m/z (ES⁺)=342.2 [M+H]⁺

### Example 84

### 4-{2-[4-(5-Methanesulfonylpyridin-2-yl)piperazin-1-yl]ethyl}piperidine-1-carboxylic acid tert-butyl ester

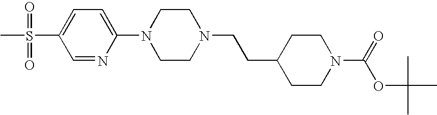

A solution of 4-(5-methanesulfonylpyridin-2-yl)piperazine-1-carboxylic acid tert-butyl ester (0.075 g, 0.23 mmol) in DCM (5 mL) was treated with TFA (0.5 mL) and the mixture stirred at rt for 4 h. DCM (30 mL) was added and the organic layer washed with 1M NaOH solution, brine, dried (MgSO₄) and the solvent removed in vacuo to yield 1-(5-methanesulfonylpyridin-2-yl)piperazine as a white solid (0.07 g, 100%). The solid (0.064 g, 0.27 mmol) was dissolved in 1:1 DCM/THF (12 mL) and treated with N-boc-piperidinyl-4-acetaldehyde (0.061 g, 0.27 mmol) and 4A molecular sieves (0.1 g) at rt. The solution was allowed to stir for 1 h then treated with NaHB(OAc)₃ (0.074 g, 0.35 mmol). The resulting solution was stirred at rt for 24 h. DCM (30 mL) was added and the organic layer washed with saturated Na₂CO₃ solution, brine, dried (MgSO₄) and the solvent removed in vacuo. The crude product was triturated with Et₂O, filtered and dried in vacuo to afford the title compound (0.043 g, 35%): RT=2.49 min; m/z (ES⁺)=453.4 [M+H]⁺

Intermediate 23: 1-(4-Methanesulfonylphenyl)-4-(2-piperidin-4-ylethyl)piperazine

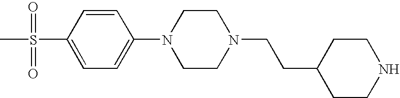

To a solution of 4-{2-[4-(4-methanesulfonylphenyl)piperazin-1-yl]ethyl}piperidine-1-carboxylic acid tert-butyl ester (5 g, 11.1 mmol) in DCM (10 mL) was added 1:3 DCM/TFA dropwise over 30 min. The reaction was stirred for an additional 30 min and the solvent removed in vacuo. The residue was dissolved in EtOAc and washed with 1M NaOH. The combined basic aqueous was saturated with NaCl and back extracted with EtOAc. The combined organic phases were washed with brine, dried (MgSO₄) and the solvent removed in vacuo to afford the title compound (2.98 g, 77%): RT=0.26 min; m/z (ES⁺)=352.1 [M+H]⁺

### Example 85

### 4-{2-[4-(4-Methanesulfonylphenyl)piperazin-1-yl]ethyl}piperidine-1-carboxylic acid propyl ester

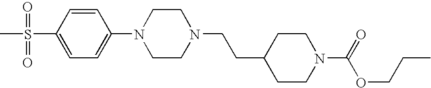

To a solution of 1-(4-methanesulfonylphenyl)-4-(2-piperidin-4-yl-ethyl)piperazine (0.05 g, 0.14 mmol) and NEt₃ (0.06 mL, 0.42 mmol) in DCM (1 mL) was added propyl chloroformate (0.019 mL, 0.17 mmol) and the mixture stirred at rt for 2 h. Water was added and the two layers separated via phase separator cartridge and the solvent removed in vacuo to afford the title compound (0.04 g, 65%): RT=2.45 min; m/z (ES⁺)=438.3 [M+H]⁺

### Example 86

### 4-{2-[4-(4-Methanesulfonylphenyl)piperazin-1-yl]ethyl}piperidine-1-carboxylic acid isopropyl ester

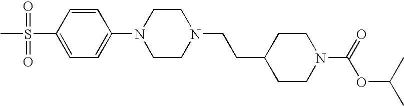

To a solution of isopropanol (0.054 mL, 0.71 mmol) and triphosgene (0.07 g, 0.24 mmol) in THF (2 mL) at 0° C. was added NEt₃ (0.2 mL, 1.42 mmol). The suspension was allowed to warm to rt over 1 h and then added to a solution of 1-(4-methanesulfonylphenyl)-4-(2-piperidin-4-ylethyl)piperazine (0.05 g, 0.14 mmol) in THF (1 mL). The mixture was stirred for 2 h and the solvent removed in vacuo. The crude solid was dissolved in DCM and washed with water, dried via phase separator and the solvent removed in vacuo to yield a crude solid which was purified by HPLC to afford the title compound (0.01 g, 16%): RT=2.45 min; m/z (ES⁺)=438.3 [M+H]⁺

### Example 87

### 4-{2-[4-(4-Methanesulfonylphenyl)piperazin-1-yl]ethyl}piperidine-1-carboxylic acid 1-methylcyclobutyl ester

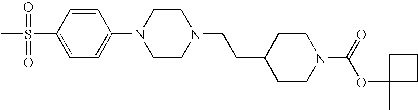

Prepared using the above method: RT=2.54 min; m/z (ES⁺)=464.4 [M+H]⁺

### Example 88

### 2-(4-{2-[4-(4-methanesulfonylphenyl)piperazin-1-yl]ethyl}piperidin-1-yl)-5-methylpyrimidine

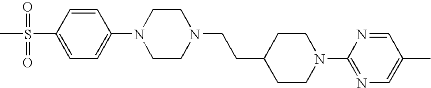

To a solution of 1-(4-methanesulfonylphenyl)-4-(2-piperidin-4-ylethyl)piperazine (0.05 g, 0.14 mmol) and DBU (0.026 mL, 0.17 mmol) in dioxane (1 mL) was added 2-chloro-5-methyl pyrimidine (0.021 g, 0.16 mmol). The mixture was stirred for 3.5 days and the solvent removed in vacuo. The crude mixture was purified by HPLC to afford the title compound (0.018 g, 29%): RT=2.24 min; m/z (ES⁺)=444.3 [M+H]⁺

### Example 89

### 5-Fluoro-2-(4-{2-[4-(4-methanesulfonylphenyl)piperazin-1-yl]ethyl}piperidin-1-yl)pyrimidine

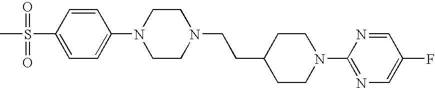

To a degassed solution of Example 85 (0.2 g, 0.57 mmol), 2-chloro-5-fluoro pyrimidine (0.076 g, 0.57 mmol), sodium tert-butoxide (0.082 g, 0.86 mmol), 9,9-dimethyl-4,5-bis(diphenylphosphino)xanthene (0.02 g, 0.032 mmol) in toluene was added Pd₂(dba)₃ (0.011 g, 0.01 mmol). The mixture was heated at reflux for 2 h, cooled and the solvent removed in vacuo. The crude mixture was purified by flash chromatography with 1% NH₃, 1% MeOH/DCM as eluent to afford the title compound (0.017 g, 7%): RT=2.51 min; m/z (ES⁺)=448.2 [M+H]⁺

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Ste3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR116 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR116 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give a increase of at least 5-fold that of the background signal may be preferred.

cAMP Assay

A stable cell line expressing recombinant human GPR116 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol. Compounds of the invention showed a concentration-dependant increase in intracellular cAMP level.

Compounds of the invention produced a concentration-dependent increase in intracellular cAMP level and generally had an EC₅₀ of <10 μM. Compounds showing an EC₅₀ of less than 1 um in the cAMP assay may be preferred.

### In Vivo Feeding Study

The effect of compounds of the invention on body weight and food and water intake may be examined in freely-feeding male Sprague-Dawley rats maintained on reverse-phase lighting. Test compounds and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage. Animals are maintained on a reverse phase light-dark cycle (lights off for 8 h from 09.30-17.30 h) during which time the room was illuminated by red light. Animals have free access to a standard powdered rat diet and tap water during a two week acclimatization period. The diet is contained in glass feeding jars with aluminum lids. Each lid has a 3-4 cm hole in it to allow access to the food. Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μl reactions were set up containing 8 μl sample, 50 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred

### Insulin Secretion Assay

HIT-T15 cells were plated in standard culture medium in 12-well plates at 106 cells/1 ml/well and cultured for 3 days and the medium was then discarded. Cells were washed ×2 with supplemented Krebs-Ringer buffer (KRB) containing 119 mM NaCl, 4.74 mM KCl, 2.54 mM CaCl₂, 1.19 mM MgSO₄, 1.19 mM KH2PO4, 25 mM NaHCO₃, 10 mM HEPES at pH 7.4 and 0.1% bovine serum albumin. Cells were incubated with 1 ml KRB at 37° C. for 30 min which was then discarded. This was followed by a second incubation with KRB for 30 min, which was collected and used to measure basal insulin secretion levels for each well. Compound dilutions (0, 0.1, 0.3, 1, 3, 10 uM) were then added to duplicate wells in 1 ml KRB, supplemented with 5.6 mM glucose. After 30 min incubation at 37° C. samples were removed for determination of insulin levels. Measurement of insulin was done using the Mercodia Rat insulin ELISA kit, following the manufacturers instructions, with a standard curve of known insulin concentrations. For each well insulin levels were subtracted by the basal secretion level from the preincubation in the absence of glucose. Data was analysed using XLfit 3 software.

### Oral Glucose Tolerance Tests

The effects of compounds of the invention on oral glucose (Glc) tolerance may be evaluated in male C57B1/6 or male ob/ob mice. Food is withdrawn 5 h before administration of Glc and remains withdrawn throughout the study. Mice have free access to water during the study. A cut is made to the animals' tails, then blood (20 μL) is removed for measurement of basal Glc levels 45 min before administration of the Glc load. The mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) are taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are added to 180 μL of Trinders glucose reagent (Sigma enzymatic (Trinder) colorimetric method) in a 96-well assay plate. After mixing, the samples are left at rt for 30 min before being read against Glc standards (Sigma glucose/urea nitrogen combined standard set).

What is claimed is:

1. A compound of formula (I): 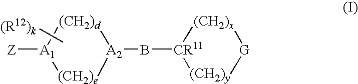
or a pharmaceutically acceptable salt thereof, wherein:
Z represents an aryl, heteroaryl, —C₁₋₄alkylaryl or —C₁₋₄alkylheteroaryl group, any of which may optionally be substituted by one or more groups selected from halogen, C₁₋₄ alkyl, C₁₋₄ fluoroalkyl, C₁₋₄ hydroxyalkyl, C₂₋₄ alkenyl, C₂₋₄ alkynyl, C₁₋₄alkoxy, OR⁹, NR³R⁴, S(O)ₙR⁹, S(O)₂NR⁹R⁹⁹, C(O)NR⁹R⁹⁹, NR¹⁰C(O)R⁹, NR¹⁰C(O)NR⁹R⁹⁹, NR¹⁰SO₂R⁹, C(O)R⁹, C(O)OR⁹, —P(O)(CH₃)₂, NO₂, cyano or —(CH₂)ⱼ—C₃₋₇ cycloalkyl, —(CH₂)ⱼ-aryl, —(CH₂)ⱼ-heterocyclyl, —(CH₂)ⱼ-heteroaryl, any of which cycloalkyl, aryl, heterocyclyl or heteroaryl groups may be substituted by C₁₋₄alkyl;
one of A₁ and A₂ is N or N⁺—O⁻, and the other is CH, C(OH) or N;
d is 0, 1, 2, or 3;
e is 1 or 2;
with the proviso that d+e is 2, 3, 4 or 5, and that if A₁ and A₂ are both N, d is 2 or 3 and e is 2;
j is 0, 1 or 2;
k is 0, 1 or 2;
n is 0, 1, or 2;
B represents a branched or unbranched C₁₋₄alkylene chain or C₁₋₄alkenylene chain, either of which may optionally be substituted by one or more groups selected from halogen, hydroxy or oxo, and wherein one CH₂ group may be replaced by O or NR⁸, provided that the group >A₂-B— does not contain any direct N—O, N—C—O, N—N, N—C—N or N—C-halogen bonds;
G represents CHR² or NR¹;
R¹ is C(O)OR⁵, C(O)R⁵, S(O)₂R⁵, C(O)NR⁵R⁸, C₁₋₄alkylene-C(O)OR⁵, C(O)C(O)OR⁵, or P(O)(O-Ph)₂; or heterocyclyl or heteroaryl, either of which may optionally be substituted by one or two groups selected from C₁₋₄alkyl, C₁₋₄alkoxy or halogen;
R² is C₃₋₆alkyl;
R³ and R⁴ are independently hydrogen, methoxy, C₁₋₄ alkyl, which may optionally be substituted by halo, hydroxy, C₁₋₄ alkyloxy-, aryloxy-, arylC₁₋₄ alkyloxy-, C₁₋₄ alkylS(O)ₙ—, C₃₋₇ heterocyclyl, —C(O)OR¹⁴ or N(R¹⁰)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR¹³, CN, SO₂CH₃, N(R¹⁰)₂ and NO₂; or taken together R³ and R⁴ may form a 5- or 6-membered heterocyclic ring optionally substituted by hydroxy, C₁₋₄ alkyl or C₁₋₄ hydroxyalkyl and optionally containing a further heteroatom selected from O and NR¹⁰;
R⁵ and R⁵⁵ are independently C₁₋₈ alkyl, C₂₋₈ alkenyl or C₂₋₈ alkynyl, any of which may be optionally substituted by one or more halo atoms, NR⁶R⁶⁶, OR⁶, C(O)OR⁶, OC(O)R⁶ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyeneheterocyclyl or C₁₋₄ alkyleneheteroaryl, any of which may be substituted with one or more substituents selected from halo, C₁₋₄ alkyl, C₁₋₄ fluoroalkyl, OR⁷, CN, NR⁷R⁷⁷, SO₂Me, NO₂ or C(O)OR⁷;
R⁶, R⁶⁶, R⁷, and R⁷⁷ each independently are hydrogen or C₁₋₄alkyl; or, taken together, R⁶ and R⁶⁶ or R⁷ and R⁷⁷ may independently form a 5- or 6-membered heterocyclic ring;
R⁸ hydrogen or C₁₋₄alkyl;
R⁹ and R⁹⁹ are independently hydrogen, methoxy, C₁₋₄ alkyl, which may optionally be substituted by halo, hydroxy, C₁₋₄ alkoxy-, C₁₋₄ alkoxyC₁₋₄ alkoxy-, -aryloxy-, arylC₁₋₄ alkyloxy-, C₁₋₄ alkylS(O)ₙ—, C₃₋₇ heterocyclyl, —C(O)OR¹⁴ or N(R¹⁰)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR¹³, CN, SO₂CH₃, N(R¹⁰)₂ and NO₂; or taken together R⁹ and R⁹⁹ may form a 5- or 6-membered heterocyclic ring optionally substituted by hydroxy, C₁₋₄ alkyl or C₁₋₄ hydroxyalkyl and optionally containing a further heteroatom selected from O and NR¹⁰;
R¹⁰ is hydrogen, C₁₋₄ alkyl; or a group N(R¹⁰)₂ may form a 4- to 7-membered heterocyclic ring optionally containing a further heteroatom selected from O and NR¹⁰;
R¹¹ is hydrogen or hydroxy, or when B represents C₁₋₄alkenylene and there is a point of unsaturation adjacent to CR¹¹ then R¹¹ is absent;
R¹² is each independently hydroxy, oxo, methyl; or two R¹² groups may form a bridging methylene;
R¹³ is hydrogen, C₁₋₂ alkyl or C₁₋₂ fluoroalkyl;
R¹⁴ is hydrogen or C₁₋₄ alkyl;
x is 0, 1, 2 or 3; and
y is 1, 2, 3, 4 or 5;
with the proviso that x+y is 2, 3, 4 or 5.

2. A compound according to claim 1, or a pharmaceutically acceptable salt thereof, wherein Z is optionally substituted phenyl.

3. A compound according to claim 1, or a pharmaceutically acceptable salt thereof, wherein Z is optionally substituted 6-membered heteroaryl.

4. A compound according to claim 1, or a pharmaceutically acceptable salt thereof, wherein G is NR¹.

5. A compound according to claim 1, or a pharmaceutically acceptable salt thereof, wherein R¹ is C(O)OR⁵, C(O)NR⁵R⁸ or heteroaryl.

6. A compound according to claim 5, or a pharmaceutically acceptable salt thereof, wherein R¹ is C(O)OR⁵

7. A compound according to claim 1, or a pharmaceutically acceptable salt thereof, wherein R⁵ is C₃₋₅alkyl optionally substituted by one or more halo atoms or cyano, and may contain a CH₂ group that is replaced by O or S, or C₃₋₅cycloalkyl optionally substituted by C₁₋₄ alkyl.

8. A compound according to claim 1, or a pharmaceutically acceptable salt thereof, of formula (Ib): 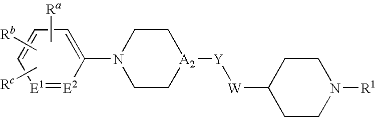
(Ib)
wherein E¹ and E² are CH, or one of E¹ and E² is N and the other is CH;
A₂ is N or CH;
when A₂ is N, Y is CH₂;
when A₂ is CH, Y is O or NR⁸;
W is a branched or unbranched C₁₋₃alkylene chain or C₁₋₃alkenylene chain, either of which may optionally be substituted by one or more groups selected from halogen, hydroxy or oxo;
one of R^{a}, R^{b} and R^{c} is selected from S(O)ₙR⁹, S(O)₂NR⁹R⁹⁹, C(O)NR⁹R⁹⁹, NR¹⁰C(O)NR⁹R⁹⁹ and 5- or 6-membered heteroaryl, and the other two of R^{a}, R^{b} and R^{c} are selected from hydrogen, halogen, C₁₋₄ alkyl and cyano; and
R¹ is C(O)OR⁵, C(O)NR⁵R⁸ or 5- or 6-membered heteroaryl.

9. A compound of formula (I) as defined in any one of Examples 1 to 89, or a pharmaceutically acceptable salt thereof.

10. A pharmaceutical composition comprising a compound according to claim 1, or a pharmaceutically acceptable salt thereof, and a pharmaceutically acceptable carrier.

11. A method for the treatment of a disease or condition in which GPR119 plays a role comprising a step of administering to a subject in need thereof an effective amount of a compound according to claim 1, or a pharmaceutically acceptable salt thereof.

12. A method for the regulation of satiety comprising a step of administering to a subject in need thereof an effective amount of a compound according to claim 1, or a pharmaceutically acceptable salt thereof.

13. A method for the treatment of obesity comprising a step of administering to a subject in need thereof an effective amount of a compound according to claim 1, or a pharmaceutically acceptable salt thereof.

14. A method for the treatment of diabetes comprising a step of administering to a subject in need thereof an effective amount of a compound according to claim 1, or a pharmaceutically acceptable salt thereof.

15. A method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to claim 1, or a pharmaceutically acceptable salt thereof.

16. (canceled)

17. (canceled)

18. (canceled)

19. A compound of formula (12): 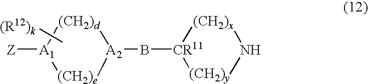
or a salt or protected derivative thereof, wherein the groups Z, A¹, A², B, R¹¹, R¹² d, e, k, x and y are as defined in claim 1.
